(19) United States
(12) Patent Application Publication
(10) Pub. No.: US 2026/0055391 A1
(43) Pub. Date: Feb. 26, 2026

(54) Genetically Encoded Tyrosine Sulfation of Proteins in Eukaryotes

(71) Applicant: Trustees of Boston College, Chestnut Hill, MA (US)

(72) Inventors: Abhishek Chatterjee, Lexington, MA (US); James S. Italia, Charlestown, MA (US)

(21) Appl. No.: 19/330,133

(22) Filed: Sep. 16, 2025

Related U.S. Application Data
(62) Division of application No. 17/594,570, now Pat. No. 12,435,327.
(60) Provisional application No. 62/837,217, filed on Apr. 23, 2019.

(51) Int. Cl. C12N 9/00; C12P 21/00

(52) U.S. Cl. CPC C12N 9/93; C12P 21/00; C12Y 601/01001

(57) ABSTRACT

An engineered tyrosyl-tRNA synthetase/tRNA pair that co-translationally incorporates O-sulfotyrosine in response to UAG codons in E. coli and mammalian cells is described herein. This platform enables recombinant expression of eukaryotic proteins homogeneously sulfated at chosen sites.

### RELATED APPLICATIONS

This application is a Divisional of U.S. application Ser. No. 17/594,570, filed on Oct. 22, 2021, which is a § 371 National Phase Application of International Application No. PCT/US2020/029567, filed on Apr. 23, 2020, now International Publication No. WO 2020/219708, published on Oct. 29, 2020, which International Application claims the benefit under 35 USC 119(e) of U.S. Provisional Application No. 62/837,217, filed on Apr. 23, 2019, all of which are incorporated herein by reference in their entirety.

### GOVERNMENT SUPPORT

The current technology was developed in part using funds supplied by the National Institutes of Health (NIH) under grant Nos. R01GM124319 and R01GM126220. Accordingly, the U.S. Government has certain rights to this invention.

### REFERENCE TO AN ELECTRONIC SEQUENCE LISTING

A Sequence Listing conforming to the rules of WIPO Standard ST.26 is hereby incorporated by reference. Said Sequence Listing has been filed as an electronic document via Patent Center encoded as XML in UTF-8 text. The electronic document, created on Sep. 8, 2025, is entitled “0342.0007US2_ST26.xml”, and is 76,289 bytes in size.

### FIELD OF THE INVENTION

The present invention is directed to genetically encoded protein sulfation in eukaryotes.

### BACKGROUND OF THE INVENTION

Sulfation of tyrosine residues is a post-translational modification (PTM) that occurs exclusively in multicellular eukaryotes. Golgi-resident tyrosylprotein sulfotransferases (TPST1 and TPST2) use 3′-phosphoadenosine-5′-phosphosulfate (PAPS) as the sulfate donor to install this PTM (FIG. 1a). Consequently, only secreted and membrane-associated proteins, which are processed through the trans-Golgi network, are subjected to this modification. Tyrosine sulfation is believed to be irreversible and it facilitates numerous protein-protein and protein-ligand interactions that are important in diverse physiological processes such as immunity, hormone function, and blood-coagulation. Additionally, pathogens such as HIV rely on tyrosine-sulfated cell-surface receptors to gain access to human cells. In turn, our immune system employs tyrosine-sulfated antibodies to target such pathogens, attesting to the importance of this PTM in our biology.

It is estimated that ˜1% of all tyrosine residues in the eukaryotic proteome are sulfated, but the physiological roles for the most remain poorly understood. A major factor contributing to this knowledge gap is the difficulty of expressing target proteins in a homogeneously sulfated state. Recombinant expression in common eukaryotic hosts often leads to incomplete sulfation of native sites. Additionally, many proteins are sulfated at multiple tyrosine residues, and the difficulty of only modifying a chosen subset of these makes it challenging to evaluate the roles of individual sulfations. Consequently, the ability to express recombinant proteins, where a specific group of tyrosine residues are sulfated is important to both understand and take advantage of the biology of protein sulfation. However, when proteins are overexpressed, the endogenous sulfotransferases cannot keep up with the high expression levels, leading to heterogeneous modification of endogenous sulfation sites. Furthermore, if multiple, distinct sulfation sites are present in a protein, it is not possible to homogenously modify a subset.

### SUMMARY OF THE INVENTION

Tyrosine sulfation is an important post-translational modification found in higher eukaryotes. The present invention is directed to genetically encoded protein sulfation in eukaryotes. Described herein are methods and compositions comprising an engineered tyrosyl RNA synthetase/tRNA pair that co-translationally incorporates tyrosine analogs, specifically O-sulfotyrosine, in response to UAG codons in Escherichia coli (E. coli) and mammalian cells. The methods and compositions described herein enable recombinant expression of eukaryotic proteins of interest that are homogeneously sulfated at selected sites (site-specific incorporation).

Compositions are described herein comprising a variant E. coli tyrosyl-tRNA synthetase (EcTyr-RS) wherein the EcTyr-RS preferentially aminoacylates an E. coli tyrosyl tRNA (Ec-tRNA^{Tyr}) with a tyrosine analog over the naturally-occurring tyrosine amino acid.

The tyrosine analog described herein (also referred to herein as a derivative) is sulfotyrosine, and more particularly O-sulfotyrosine. Other tyrosine analogs suitable for use as described herein can be synthesized by one of skill in the art using known methods.

In particular, the current invention encompasses a composition comprising a variant E. coli tyrosyl-tRNA synthetase (EcTyr-RS) wherein the variant EcTyr-RS comprises the 424 amino acid sequence of E. coli published in the NCBI database for the K-12 E. coli strain, substrain DH10B (ncbi.nlm.nih.gov/protein and Gen Bank accession number ACB02843) or E. coli strain MG1655 NCBI Reference Sequence NP_416154.1 (SEQ ID NO:1) which preferentially aminoacylates an E. coli tyrosyltRNA (EctRNA^{tyr}) with a tyrosine analog over the naturally-occurring tyrosine amino acid, wherein the variant EcTyr-RS comprises the amino acid sequence of SEQ ID NO: 1, or a homologous EcTyr-RS comprising an amino acid sequence with at least about 80%, about 85%, about 90%, about 95% or up to about 99% sequence identity with the full-length SEQ ID NO: 1 and comprising mutated amino acid positions. More specifically, engineered/mutated variants of the EcTyr-RS comprise the EcTyr-RS wherein the EcTyr-RS, or homologous EcTyr-RS, is mutated/modified at one, or more active site residues. EcTyr-RS mutants with the ability to selectively recognize O-sulfotyrosine were selected from a large mutant library, where the following actives site residues were randomized using the design as follows: the tyrosine (Y) is replaced with FLIMVSTAYHCG (SEQ ID NO: 2) at position 37; the leucine (L) is replaced with NBT at position 71; the asparagine (N) is replaced with NSPTACGDH (SEQ ID NO: 3) at position 126; the aspartic acid (D) is replaced with NST at position 182; the phenylalanine (F) is replaced with NNK at position 183; or the leucine (L) is replaced with NNK at position 186. Polynucleotide sequences encoding these proteins/polypeptides are also encompassed herein.

Specifically encompassed by the present inventions are the following EcTyr-RNA synthetase mutants, identified from the selection of a library of library of ˜10⁷ mutants described below, wherein the EcTyr-RS comprises the amino acid sequence SEQ ID NO: 1 wherein the tyrosine (Y) at position 37, and the asparagine (N) at position 126 are conserved, the leucine (L) at position 71 is replaced with valine (V), the aspartic acid (D) at position 182 is replaced with G, the phenylalanine at position 183 is either conserved or mutated to Y, and the L at position 186 is either conserved, or is replaced with M, I or V. The exact sequences of each EcTyr-RS mutants are provided herein as SEQ ID NOS; 4-9. The VGL clone (SEQ ID NO: 4) is also referred to herein as the EcTyr-RS mutant VGL and the VGM clone (SEQ ID NO: 5) is referred to herein as the EcTyr-RS mutant VGM. Additional EcTyrRS variants are the EcTryRS mutant VGYI (SEQ ID NO: 6); EcTyrRS mutant VGYL (SEQ ID NO:7); EcTryRS mutant VGYLR (SEQ ID NO: 8) and EcTyrRS mutant VGV (SEQ ID NO:9) as shown in FIGS. 16A-G.

The Tyr-RNA synthetases encompassed by the present invention further include homologous bacteria-derived Tyr-RNA synthetases with active-site residues substituted with mutations as described herein. Such homologous TyrRS genes can be identified by techniques known to those of skill in the art, for example by performing sequence identity/homology searches of TyrRS genetic sequence databases to identify TyrRS gene sequences with, for example, about 80% sequence identity; about 85% sequence identity; about 90% sequence identity; about 95% sequence identity or greater than about 95% sequence identity, which are substantially homologous, or highly homologous to the E. coli TyrRS described herein. Such homologous TyrRS genes suitable for use as described herein may contain sequence variation from the E. coli Tyr-RS wherein such sequence variations do not affect the functionality (aminoacyl activity) of the RNA synthetase. Such nucleotide variations can also be defined as conservative sequence variations or substitutions. Also encompassed by the present invention are complementary polynucleotide sequences and polynucleotide sequences that hybridize under highly stringent conditions over substantially the entire length of the nucleotide sequence, as well as the polypeptides encoded by the polynucleotides. The homologous bacteria-derived Tyr-RS can be mutated at its active-site residues corresponding to the mutations as described herein for the E. coli Tyr-RS.

The present invention further encompasses tRNA compositions wherein the tRNA anti-codon loop is modified (e.g., mutated) to specifically bind to (e.g., recognize) an amber (UAG/TAG) codon as described herein. tRNA compositions comprising the ochre codon (TAA/UAA) or the opal codon (UGA/TGA) are also encompassed by the present invention. In particular, the present invention encompasses compositions wherein the tRNA is the E. coli tyrosyl tRNA, or another homologous bacteria-derived tRNA, wherein the polynucleotide sequence comprises SEQ ID NO: 10 of FIG. 16H (also see for example Cell Chem. Biol. 25, 13-4-1312 (2018) (or with about 80%; about 85%; about 90%, about 95% or greater than about 95% sequence identity) with an anti-codon loop comprising a sequence that specifically binds to a selector sequence of an mRNA selected from the group consisting of an amber codon. Importantly, the tRNA EcTyr UAG described herein is a novel amber suppressor suitable for use in both genetically-engineered bacteria and eukaryotes.

It is important to note that the modified tRNA of E. coli, or a homologous bacteria-derived tRNA, can be combined with an RNA synthetase of another homologous bacteria-derived RNA synthetase to produce novel combinations for unnatural amino acid, e.g., tyrosine analog, incorporation into proteins. Additionally, a combination of two distinct tRNA-RS/tRNA pairs can be combined. For example, the EcTyr-RS/tRNA pair described can also be combined with other suitable tRNA-RS/tRNA pairs which respond to other condons such as an opal suppressor, to site-specifically incorporate two distinct unnatural amino acids into polypeptide/proteins expressed in eukaryotic cells.

Also encompassed by the present invention are cells (either cultured in vitro or in vivo) comprising an orthogonal E. coli tyrosyl-tRNA synthetase (EcTyr-RS), wherein the EcTyr-RS preferentially aminoacylates an E. coli tyrosyl tRNA with a tyrosine analog, and an orthogonal E. coli tyrosyl tRNA (Ec-tRNA^{Tyr}) as a pair. Importantly, the orthogonal TyrRS/tRNA pair) does not cross-react the cell's endogenous TyrRS/tRNA pair. Such cells comprise not only the RS/tRNA pairs described herein, but also all cellular components required for translation of polynucleotides into proteins, including translation system components such as, for example, ribosomes, endogenous tRNAs, translation enzymes, mRNA and amino acids.

The cells of the present invention can be any bacterial cell or eukaryotic cell suitable for use with the tRNA synthetase/tRNA pairs described herein. In particular, the cell can be a mammalian cell. In particular, the bacterial cell is a genetically-engineered E. coli cell, or a homologous/analogous bacterial cell. More specifically, the E. coli is the ATMY strain of E. coli cell.

A cell(s) of the present invention encompasses a cell comprising a variant E. coli tryrosyl tRNA synthetase (EcTyr-RS), wherein the variant EcTyr-RS preferentially aminoacylates an E. coli tyrosyl tRNA with a tyrosine analog, and an orthogonal E. coli tyrosyl tRNA (Ec-tRNA^{Tyr}) as a pair, wherein the variant EcTyr-RS comprises the amino acid sequence of SEQ ID NO: 1, or an amino acid sequence with at least 90% sequence identity with the full-length SEQ ID NO:1, wherein the variant E. coli EcTyr-RS is mutated, relative to SEQ ID NO:1, wherein the tyrosine (Y) at position 37, and the asparagine (N) at position 126 are conserved, the leucine (L) at position 71 is replaced with valine (V), the aspartic acid (D) at position 182 is replaced with G, the phenylalanine at position 183 is either conserved or mutated to Y, and the L at position 186 is either conserved, or is replaced with M, I or V. The exact sequences of each EcTyr-RS mutant are provided as SEQ ID NOS: 4-9 as shown in FIG. 16A-G. More particularly, the variant EcTry-RS is either the EcTyr-RS mutant VGL (SEQ ID NO: 4) or the EcTyr-RS mutant VGM (SEQ ID NO: 5).

The cell(s) of the present invention further comprise variant/mutant Ec-tRNA^{Tyr} herein (for example, SEQ ID NOS: 10) or a homologous bacteria-derived tRNA comprising at least about 80% sequence identity with SEQ ID NO: 10 or wherein the tRNA has an anti-codon loop comprising a sequence that specifically binds to a selector sequence of an mRNA, wherein the selector is an amber codon UAG.

The cell of the invention can be a bacterial cell such as an E. coli cell (e.g., ATMY, and particularly ATMY4) or a eukaryotic cell. More specifically the eukaryotic cell is a mammalian cell.

Also encompassed by the present invention are methods of producing a polypeptide/protein in a cell with one, or more, unnatural amino acids incorporated into the polypeptide/protein in a site-specific manner by one, or more of the RS/tRNA pairs described herein. In particular encompassed herein are EcTyrRS as comprised in SEQ ID NOS: 4-9. Such proteins can be labeled or chemically modified for further post-translational site-specific modifications.

Specifically encompassed by the present invention is a method of incorporating tyrosine analogs, more specifically sulfotyrosine analogs, at specified positions in a protein of interest expressed in the cell, the method comprising culturing the cell in a culture medium under conditions suitable for growth, wherein the cell comprises a nucleic acid that encodes a protein with one, or more, selector codons (e.g., amber selector codons), wherein the cell further comprises an Ec-tRNA^{Tyr} that recognizes the selector codon(s), and wherein the cell further comprises an EcTyr-RS that preferentially aminoacylates the Ec-tRNA^{Tyr} with a tyrosine analog. The cell culture medium containing the growing cells is then contacted with one, or more, tyrosine analogs under conditions suitable for incorporation of the one, or more, tyrosine analogs into the protein in response to the selector codon(s), thereby producing the protein with one, or more tyrosine analogs. The method specifically encompasses the use of the EcTyr-RS and the Ec-tRNA^{Tyr} pair described herein. Such tyrosine analogs can be sulfonated tyrosine analogs and more specifically O-sulfotyrosine, or other suitable tyrosine analogs.

Also encompassed by the present invention are methods of incorporating two, or more unnatural amino acids at specified positions in a polypeptide/protein expressed in a cell. In these methods the cell further comprises a second tRNA/RS pair that is orthogonal to the cell, wherein the second pair recognizes a selector codon in the protein but does not cross-react with the first RS/tRNA pair (e.g., EcTyr-RS/tRNA^{tyr}). The method is performed as above (or in a similar manner) wherein the protein expressed/produced contains one, or more tyrosine analogs and one, or more, distinct unnatural amino acid other than a tyrosine analog incorporated by the first RS/tRNA pair.

Also encompassed by the present invention are methods of producing a polypeptide/protein in a cell with one, or more, unnatural amino acids incorporated into the polypeptide/protein in a site-specific manner by one, or more of the RS/tRNA pairs described herein. Such proteins can be labeled or chemically modified for further post-translational site-specific modifications.

Specifically, encompassed herein are methods of producing a protein in a cell with one, or more, tyrosyl analogs at specified positions in the protein, the method comprising culturing the cell in a culture medium under conditions suitable for growth, wherein the cell comprises a nucleic acid that encodes a protein with one, or more, amber selector codons, and wherein the cell further comprises an Ec-tRNA^{Tyr} that recognizes the amber selector codon(s), and contacting the cell culture medium with one, or more, tyrosyl analogs under conditions suitable for incorporation of the one, or more, tyrosyl analogs into the protein in response to the selector codon, thereby producing the protein with one, or more tyrosyl analogs.

Mores specifically encompassed herein is a method of site-specifically incorporating one, or more, suflotyrosine residues into a protein or peptide in a cell, the method comprising, culturing the cell in a culture medium under conditions suitable for growth, wherein the cell comprises a nucleic acid that encodes a protein or peptide of interest with one, or more, amber selector codons at specific sites in the protein or peptide, wherein the cell further comprises a variant E. coli tyrosyl-tRNA synthetase (EcTyr-RS), wherein the EcTry-RS preferentially aminoacylates an E. coli tyrosyl tRNA (Ec-tRNA^{Try}) that recognizes the amber selector codon, and contacting the cell culture medium with one, or more, sulfotyrosine residues under conditions suitable for incorporation of the one, or more, sulfotyrosine residues into the protein or peptide at the sites of the selector codon(s), hereby producing the protein or peptide of interest with one, or more site-specifically incorporated sulfotyrosine residues.

These methods described above comprise the variant E. coli tyrosyl-tRNA synthetase (EcTyr-RS) that comprises the amino acid sequence of SEQ ID NO: 1, or an amino acid sequence with at least 90% sequence identity with the full-length SEQ ID NO: 1, wherein the variant EcTyr-RS preferentially aminoacylates an E. coli tyrosyl tRNA with a tyrosine analog, and an orthogonal E. coli tyrosyl tRNA (Ec-tRNA^{Tyr}) as a pair, wherein the variant EcTyr-RS comprises the amino acid sequence of SEQ ID NO: 1, or an amino acid sequence with at least 90% sequence identity with the full-length SEQ ID NO: 1, wherein the variant E. coli EcTyr-RS is mutated, relative to SEQ ID NO: 1, wherein the tyrosine (Y) at position 37, and the asparagine (N) at position 126 are conserved, the leucine (L) at position 71 is replaced with valine (V), the aspartic acid (D) at position 182 is replaced with G, the phenylalanine at position 183 is either conserved or mutated to Y, and the L at position 186 is either conserved, or is replaced with M, I or V. The exact sequences of each EcTyr-RS mutant are provided herein as SEQ ID NOS: 4-9. More particularly, variant EcTry-RS is either the EcTyr-RS mutant VGL (SEQ ID NO: 4) or the EcTyr-RS mutant VGM (SEQ ID NO: 5).

The methods described above also comprise the Ec-tRNA^{Tyr} polynucleotide sequence comprises SEQ ID NO: 10, or a homologous bacteria-derived tRNA comprising at least about 80% sequence identity with SEQ ID NO:10, wherein the tRNA has an anti-codon loop comprising a sequence that specifically binds to a selector sequence of an mRNA, wherein the selector sequence is an amber, ochre or opal codon (more specifically the amber codon). The cell(s) of the methods described herein can be bacterial cells such as an E. coli cell, or a eukaryotic cell, such as a mammalian cell. In particular the E. coli cell can be the ATMY4 strain of E. coli cell.

The methods described above encompass cells that further comprise a second tRNA/RS pair that is orthogonal to the cell, wherein the second pair does not cross-react with the EcTyr-RS/tRNA pair and that recognizes a selector codon in the protein, wherein the protein or peptide of interest produced contains one, or more sulfotyrosyl residues and one, or more, distinct unnatural amino acid residues other than a sulfotyrosyl residue.

Also encompassed by the present invention are kits for producing a protein or peptide of interest in a cell, wherein the protein or peptide comprises one, or more tyrosyl analogs, the kit comprising a container containing a polynucleotide sequence encoding an Ec-tRNA^{Tyr}) that recognizes an amber selector codon in a nucleic acid of interest in the cell and a container containing a variant E. coli tyrosyl tRNA synthetase that preferentially aminoacylates the Ec-tRNA^{Tyr} with a tryrosyl analog, wherein the EcTry-RS comprises the amino acid sequence of SEQ ID NO: 1, or an amino acid sequence with at least 90% sequence identity with the full-length SEQ ID NO:1, wherein the variant EcTyr-RS preferentially aminoacylates an E. coli tyrosyl tRNA with a tyrosine analog, and an orthogonal E. coli tyrosyl tRNA (Ec-tRNA^{Tyr}) as a pair, wherein the variant EcTyr-RS comprises the amino acid sequence of SEQ ID NO: 1, or an amino acid sequence with at least 90% sequence identity with the full-length SEQ ID NO:1, wherein the variant E. coli EcTyr-RS is mutated, relative to SEQ ID NO:1, wherein the tyrosine (Y) at position 37, and the asparagine (N) at position 126 are conserved, the leucine (L) at position 71 is replaced with valine (V), the aspartic acid (D) at position 182 is replaced with G, the phenylalanine at position 183 is either conserved or mutated to Y, and the L at position 186 is either conserved, or is replaced with M, I or V. The exact sequences of each EcTyr-RS mutant are provided as SEQ ID NOS; 4-9. More particularly, variant EcTry-RS is either the EcTyr-RS mutant VGL (SEQ ID NO:4) or the EcTyr-RS mutant VGM (SEQ ID NO: 5).

The kit can further comprise one, or more, tyrosyl analogs, in particular wherein the tyrosyl analog is sulfotyrosine. The kit can also comprise instructions for producing the protein or peptide of interest.

The present invention, as described herein, enables the expression of eukaryotic proteins in eukaryotic cells with precisely installed sulfation, such that the consequences of protein sulfation can be studied. Sulfotyrosine is a nonhydrolyzable structural mimic of phosphotyrosine which is a ubiquitous post translational modification that regulates numerous signal transduction pathways. Normally, intracellular proteins are not sulfated. However, as a result of the compositions and methods described herein, sulfotyrosine can be incorporated at known tyrosine phosphorylation sites. Sulfotyrosine can mimic the consequences of tyrosine phosphorylation due to its structural similarity, but this modification cannot be removed by endogenous phosphatases, resulting in a powerful new way to uncover the role of distinct phosphorylations in eukaryotic biology.

Many therapeutically relevant proteins are sulfated, where the sulfation is functionally important such as, e.g., in IgG and Factor IX. As a result of the compositions and methods described herein, such expression of proteins homogeneously incorporating sulfotyrosine at the desired site is now possible. Many viruses and pathogens use sulfotyrosine or sulfated sugars (e.g., heparin sulfate proteoglycan) as receptors for cellular entry. Our immune system produces sulfated antibodies that take advantage of these sulfate-binding pockets for installing a sulfation that restricts what kind of sequences can be present in the variable region of a sulfated antibody, thus limiting the sequence space accessible to such antibodies. The compositions and methods described herein would enable sulfation of any sequence contexts, which would overcome this limitation and the development of sulfated antibodies with enhanced pathogen targeting. Additionally, the methods and compositions of the present invention enable the development of antibodies that target specific sulfation sites in specific peptide contexts.

The current invention demonstrates features and advantages that will become apparent to one of ordinary skill in the art upon reading the attached Detailed Description.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. The patent or application file contains at least one drawing executed in color. Copies of this patent or patent application publication with color drawing) s) will be provided by the Office upon request and payment of the necessary fee. Of the drawings:

FIG. 1A-B shows tyrosine sulfation. 1a, Proteins processed through the trans-Golgi network in multicellular eukaryotes are subjected to tyrosine sulfation by TPST enzymes that use PAPS as a cofactor. 1b, A sulfotyrosine residue can be co-translationally incorporated into proteins expressed in living cells in response to a nonsense codon using an engineered TyrRS/tRNA pair.

FIG. 2A-G shows genetically encoding sTyr using the EcTyrRS/tRNA pair. 2a, The active site of EcTyrRS showing the bound substrate in magenta, and highlighting residues that were randomized. Mutations found in the sTyr selective variants are noted in parenthesis in red. 2b, Two EcTyrRS mutants facilitate sfGFP-151-TAG reporter expression in ATMY4 E. coli in the presence of sTyr (fluorescence in resuspended cells). 2c, ESI-MS analysis of the purified sfGFP-151-sTyr show expected mass. 2d, Expression of EGFP-39-TAG reporter in HEK293T cells using VGM-EcTyrRS/tRNA in the presence and absence of sTyr (fluorescence microscopy image). 2e, EGFP-39-TAG expression in HEK293T cell using VGL- and VGM-EcTyrRS (fluorescence in clarified cell-free extract). 2f, ESI-MS analysis of the purified EGFP-39-sTyr show expected mass. 2g, Purified wild-type and sTyr-incorporated sfGFP (ATMY4-expressed) and EGFP (HEK293T-expressed) reporter proteins analyzed by anti-sTyr and anti-polyhistidine tag Western blot, as well as Coomassie staining following SDS-PAGE. Data in e and f shown as mean±s.d. (n=3 independent experiments)

FIG. 3A-B shows expression and biochemical analysis of precisely sulfated HCII. 3a, The model for GAG-activated thrombin inhibition of HCII, which is sulfated at Tyr60 and Tyr73 (shown as green stars). 3b, Second-order rate constant of thrombin inhibition by different HCII mutants at various heparin concentrations.

FIG. 4A-B shows examples of non-naturally occurring amino acids (ncAAs) that can be reasonably genetically encoded in E. coli using, for example, the MjTyrRS/tRNA pair (4a). 4b shows ncAAs that can be reasonably genetically encoded in eukaryotes using the EcTyrRS/tRNA pair s described herein.

FIG. 5A-B show the bacteria-derived aaRS/tRNA pairs (color-coded red) are orthogonal in eukaryotes and can be used for eukaryotic genetic code expansion, while eukaryote or archaea derived pairs (color-coded blue) are orthogonal in bacteria and are useful for bacterial genetic code expansion. 5b, Functionally substituting the EcTyrRS/tRNA pair in E. coli with the archaea derived MjTyrRS/tRNA pair creates an engineered ATMY strain. The ‘liberated’ EcTyrRS/tRNA pair can be established as an orthogonal nonsense suppressor in ATMY E. coli and engineered in this strain for altering its substrate specificity.

FIG. 6A-B show the pool of EcTyrRS library of mutants selected through a single round each of positive and negative selection show substantial sTyr-dependent survival in a subsequent round of positive selection. 6b, shows that many individual clones isolated from these plates also show the same phenotype.

FIG. 7A-B show fluorescence images of HEK293T cells expressing EGFP-39-TAG reporter using VGL- or VGM-EcTyrRS mutant in the presence or absence of sTyr (1 mM).

FIG. 8 shows SDS-PAGE analysis of secreted HCII mutants expressed in HEK293T cells and isolated from the culture media using a C-terminal polyhistidine tag. Due to well-established glycosylations, the observed molecular weight is significantly larger than what is predicted from the primary sequence (˜57 kDa).

FIG. 9 shows trypsin digestion followed by LC-MS analysis of HCII-60-sTyr-73-sTyr isolated from HEK293T cells identifies the presence of the peptide harboring 60-sTyr (ENTVTNDWIPEGEEDDDY*LDLEK SEQ ID NO:11).

FIG. 10 shows trypsin+elastase double digestion followed by LC-MS analysis of HCII-60-sTyr-73-sTyr isolated from HEK293T cells identifies the presence of the peptide harboring 73-sTyr (FSEDDDY*IDIV SEQ ID NO: 13). The HCII fragment harboring the 73 residue through trypsin digestion alone was not found, likely due to its large predicted size.

FIG. 11 shows PNGase F treatment of purified HCII-60-sTyr-73-sTyr substantially reduces its molecular weight by removing N-linked glycans.

FIG. 12 depicts the plasmid map of pB1U-Sulfo-16xtYR-TAG.

FIG. 13A-E shows the continuous sequence of plasmid pB1U-Sulfo-16xtYR-TAG (SEQ ID NO:15.

FIG. 14 depicts the plasmid map of pB3-sulfoRS-16xtR-R265-HCIIx.

FIG. 15A-G shows the continuous sequence of plasmid pB3-sulfoRS-16xtR-R265-HCIIx (SEQ ID NO:16).

FIG. 16A depicts SEQ ID NO: 1, the EcTry-RS wild type. FIGS. 16B-G depict the sequences of the variant EcTryRS (SEQ ID NOS: 4-9) and a tyrosyl tRNA (FIG. 16H, SEQ ID NO:10).

FIG. 17 shows the sulfotyrosine charging activity of the EcTyr-RS mutants VGYI; VGYL; VGYLR and VGV (SEQ ID NOS: 6-9) and VGM (SEQ ID NO:5). EcTyrRS mutants facilitate sfGFP-151-TAG reporter expression in ATMY4 E. coli in the presence of sTyr (fluorescence in resuspended cells).

### DETAILED DESCRIPTION OF THE PREFERRED EMBODIMENTS

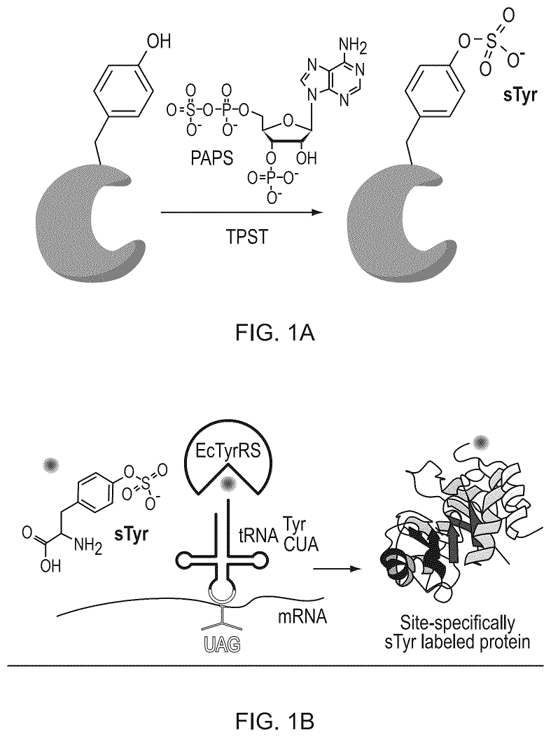

The genetic code expansion technology offers an elegant solution to challenges of producing sulfonated proteins by enabling co-translation site-specific incorporation of modified amino acid residues such as O-sulfotyrosine (sTyr) in response to a repurposed nonsense codon (FIG. 1A-B). Indeed, the M. jannaschii tyrosyl-tRNA synthetase (MjTyrRS)/tRNA pair has been engineered to site-specifically incorporate sTyr into proteins expressed in E. coli, which has been useful for investigating the roles of tyrosine sulfation. However, the MjTyrRS/tRNA pair is cross-reactive with its eukaryotic counterparts and cannot be used for non-canonical amino acid (ncAA) mutagenesis in eukaryotic cells. This significantly limits the utility of this platform, given that tyrosine sulfation is only found in proteins from multicellular eukaryotes, and that the class of eukaryotic proteins that are subjected to sulfation (secreted and membrane-associated proteins) are frequently incompatible with recombinant expression in E. coli, as they require specialized processing through the ER-Golgi network. Furthermore, the ability to express a eukaryotic protein in its native host is indispensable for investigating how its sulfation affects the cellular pathways it participates in (e.g., how sulfation of GPCRs affect their signaling). Genetically encoding sTyr in eukaryotic cells would overcome these limitations.

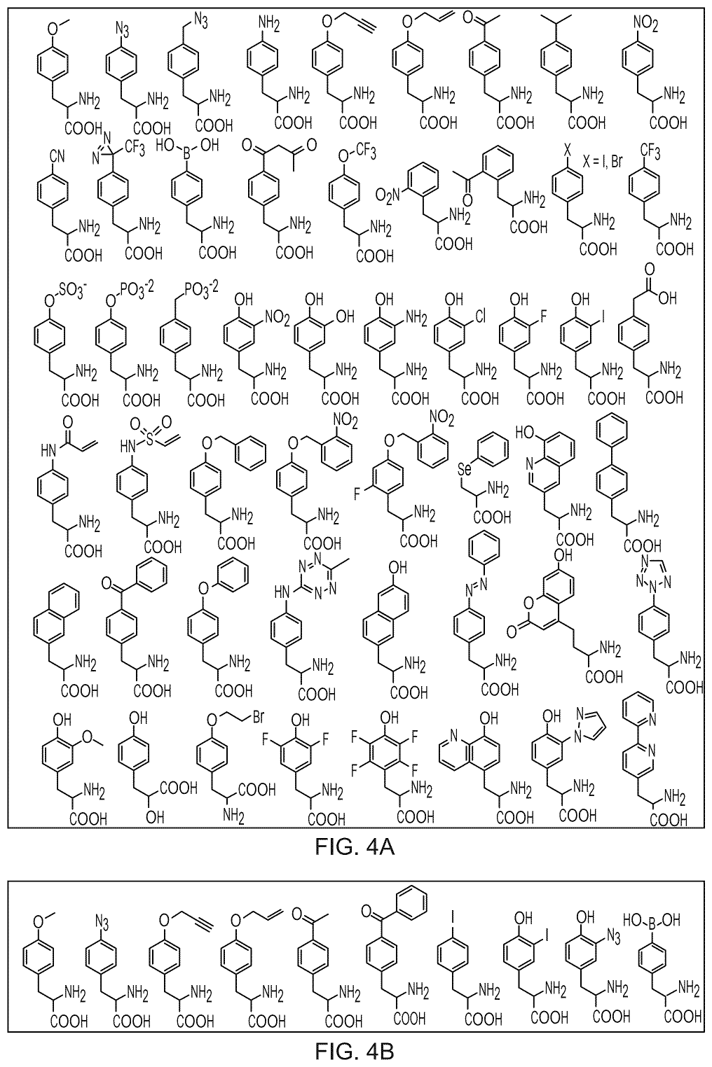

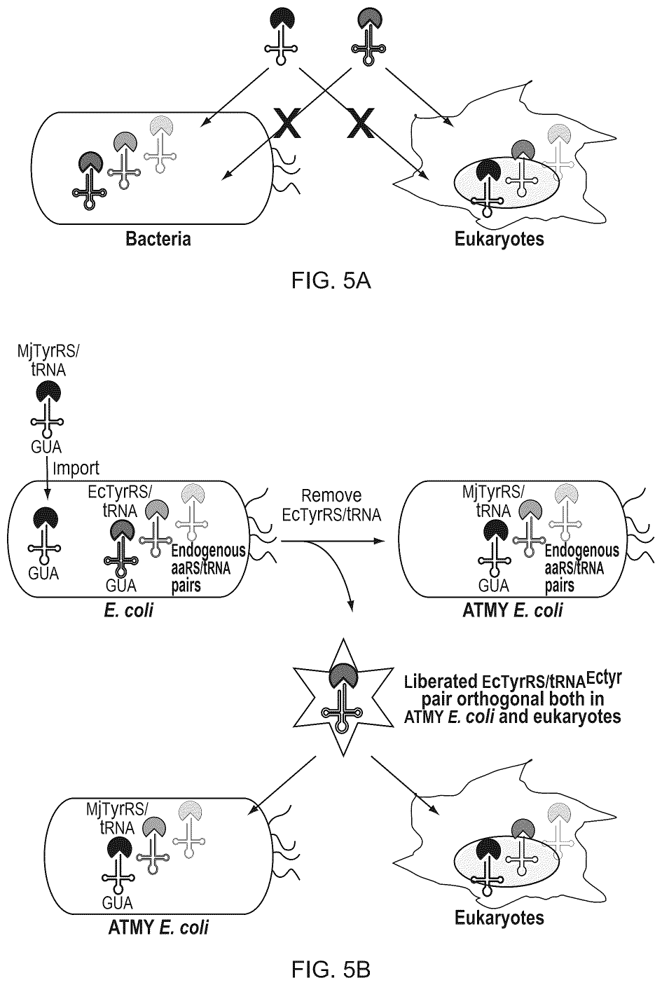

The E. coli derived tyrosyl-tRNA synthetase (EcTyrRS)/tRNA pair represents a promising platform to this end, as it has already been established for ncAA mutagenesis in eukaryotes. However, the repertoire of ncAAs genetically encoded using this platform has been significantly limited relative to its M. jannaschii derived counterpart (FIG. 4). While the substrate specificity of MjTyrRS can be engineered using a facile E. coli based directed evolution system, the engineering of EcTyrRS relies on a cumbersome yeast-based system, which has experienced much less success. Recently, a novel approach has been established to facilitate the directed evolution of E. coli derived aminoacyl-tRNA synthetase (aaRS)/tRNA pairs in E. coli (FIG. 5). First, one of the endogenous aaRS/tRNA pairs of E. coli is functionally substituted by an orthogonal counterpart from archaea/eukaryote. Next, the liberated endogenous pair is reintroduced in the resulting ‘altered translational machinery’ (ATM) E. coli strain as an orthogonal nonsense suppressor, where it can be engineered using the E. coli based directed evolution platform. This strategy has been used to create ATMY strains of E. coli, in which the endogenous EcTyrRS/tRNA pair is functionally replaced by an archaea-derived TyrRS/tRNA pair (FIG. 5b). The feasibility of engineering the EcTyrRS/tRNA pair has been further demonstrated in such an ATMY E. coli strains to genetically encode ncAAs in both eukaryotes and ATMY E. coli strains. This platform provides an exciting opportunity to genetically encode sTyr in eukaryotic cells.

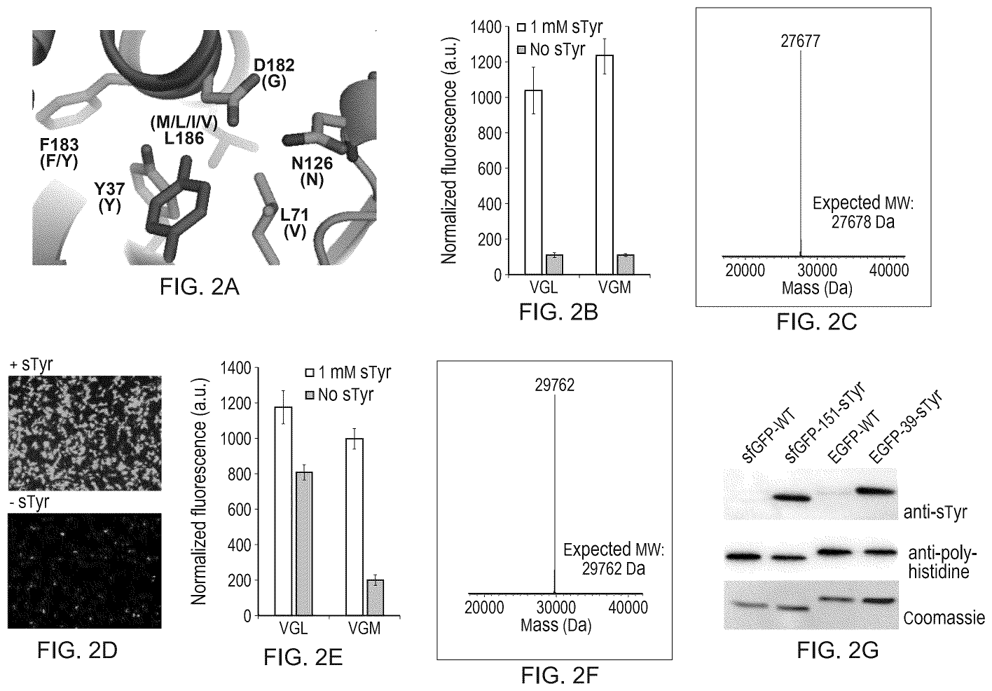

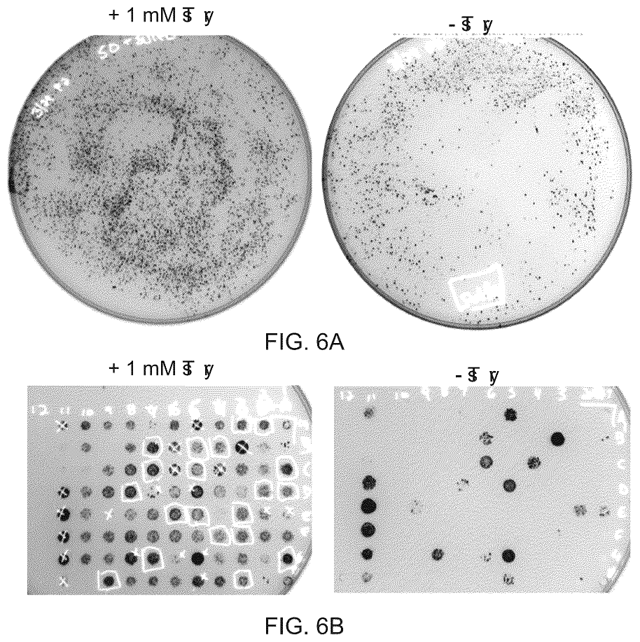

A library of EcTyrRS mutants encoded in the pBK vector (pBK-EcYRS1) was constructed by randomizing six active site residues (Y37 to FLIMVSTAYHCG (SEQ ID NO: 2), L71 to NBT, N126 to NSPTACGDH (SEQ ID NO: 3), D182 to NST, F183 to NNK, L186 to NNK) surrounding the phenolic hydroxyl of the bound tyrosine substrate (FIG. 2a). The pBK-EcYRS1 library was subjected to a previously developed double-sieve selection system in ATMY E. coli. The positive selection enriches active aaRS mutants using a TAG-inactivated chloramphenicol acetyltransferase reporter, while the negative selection removes mutants that charge canonical amino acids using a TAG-inactivated toxic barnase gene. Just after a single round of positive and negative selection each in ATMY3 E. coli, the library demonstrated highly sTyr-dependent survival in the presence of chloramphenicol, indicating the enrichment of sTyr-selective EcTyrRS mutants (FIG. 6a). Several individual clones from this selected pool of mutants also replicated the same sTyr-dependent phenotype (FIG. 6b). DNA sequencing of such clones revealed the presence of several distinct but highly convergent clones, where Y37, and N126 are conserved, L71 and D182 are mutated to V and G, respectively, F183 is either conserved or mutated to Y, and L186 is either conserved or is mutated to M, I, or V (FIG. 2a). The enlarged active sites of these mutants were consistent with the need to accommodate the additional sulfate group of sTyr.

To evaluate the sulfotyrosine incorporation efficiency of the EcTyrRS mutants the VGL and VGM (SEQ ID NO. 4 and 5, respectively) variants were were individually co-transformed (encoded in the pBK plasmid) with a pEvolT5-sfGFP-151-TAG reporter plasmid in ATMY4 E. coli strain (encodes two genomic copies of tRNAEcTyrCUA). These cells expressed sfGFP only in the presence of sTyr upon induction with IPTG (FIG. 2b). Purification of this reporter protein using a C-terminal polyhistidine tag (8-10 mg/L) followed by ESI-MS analysis showed a mass consistent with the incorporation of sTyr (FIG. 2c). Western-blot analysis using an anti-sTyr monoclonal antibody further corroborated the presence of sTyr in this protein (FIG. 2g). These observations confirm that an engineered EcTyrRS/tRNA pair that selectively incorporates sTyr in response to UAG has been generated.

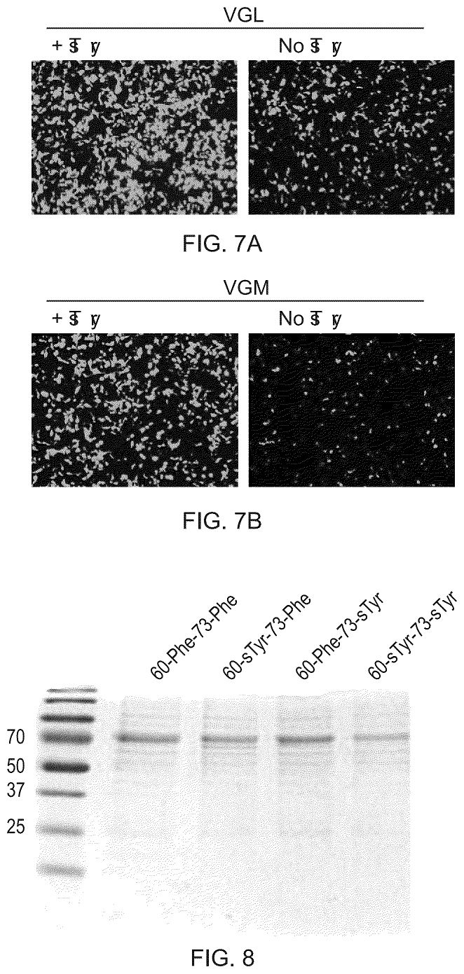

Next, it was explored if these mutant EcTyrRS/tRNA pairs can be used in mammalian cells for co-translational sTyr incorporation. A mammalian expression plasmid pB1U-Sulfo-16xtYR-TAG was created that expresses the VGL or the VGM EcTyrRS as well as 16 copies of the tRNA^{EcTyr}_{CUA}. Co-transfection of this plasmids with pAcBac1-EGFP-39-TAG led to robust expression of EGFP in the presence of sTyr, while significantly reduced reporter expression was observed in its absence (FIG. 2d-2e, FIG. 7). The VGM mutant exhibited lower levels of UAG suppression in the absence of sTyr (FIG. 2d-2e, FIG. 7). The reporter protein expressed in the presence of sTyr was isolated from HEK293T cells (100-120 μg from ˜10⁷ cells) using a C-terminal polyhistidine tag and analyzed by ESI-MS, which showed a mass consistent with sTyr incorporation (FIG. 2f). Western-blot using an anti-sTyr antibody further confirmed the presence of sTyr in EGFP-39-sTyr, but not in wild-type EGFP (FIG. 2g) that was expressed and purified in a similar manner.

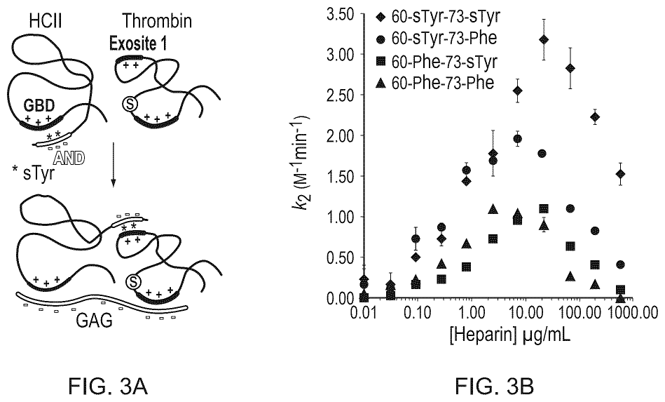

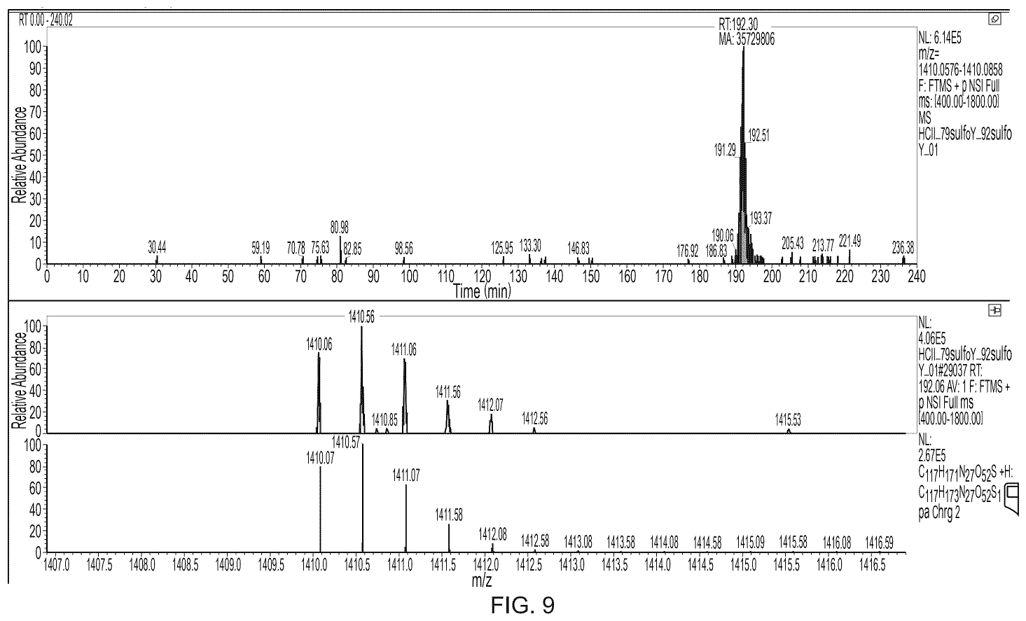

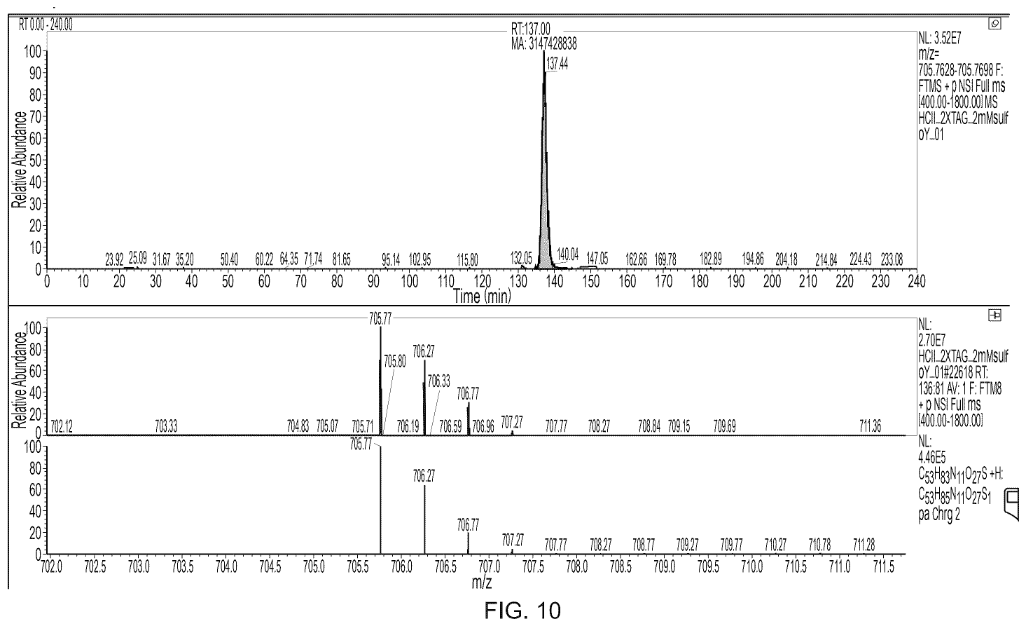

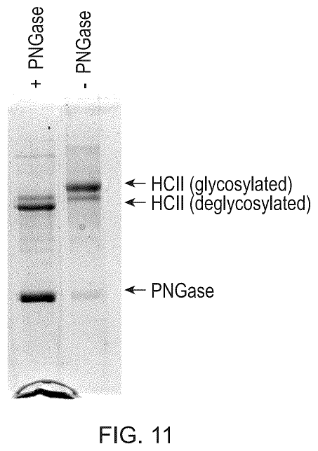

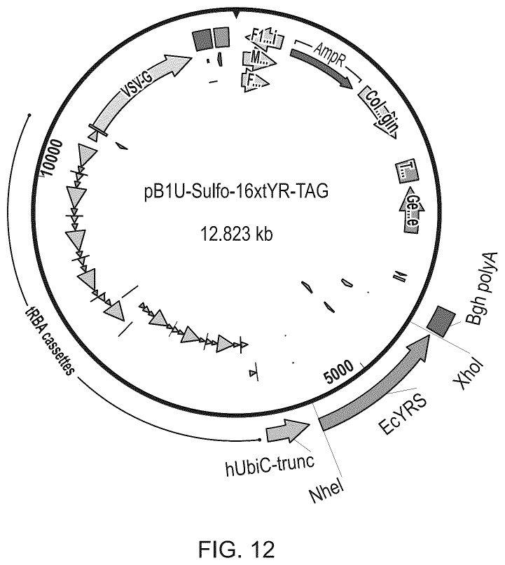

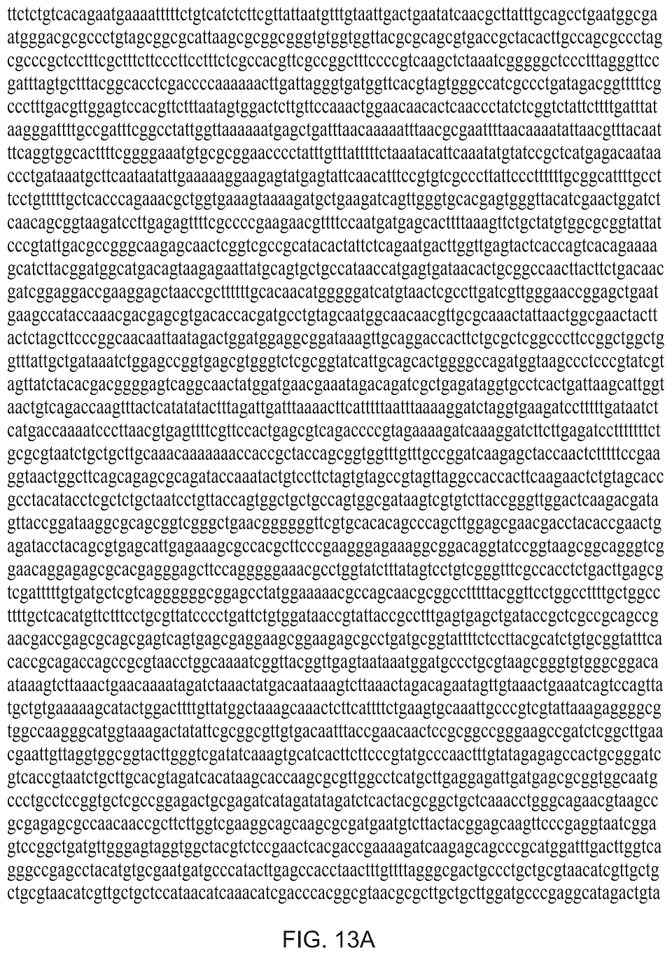

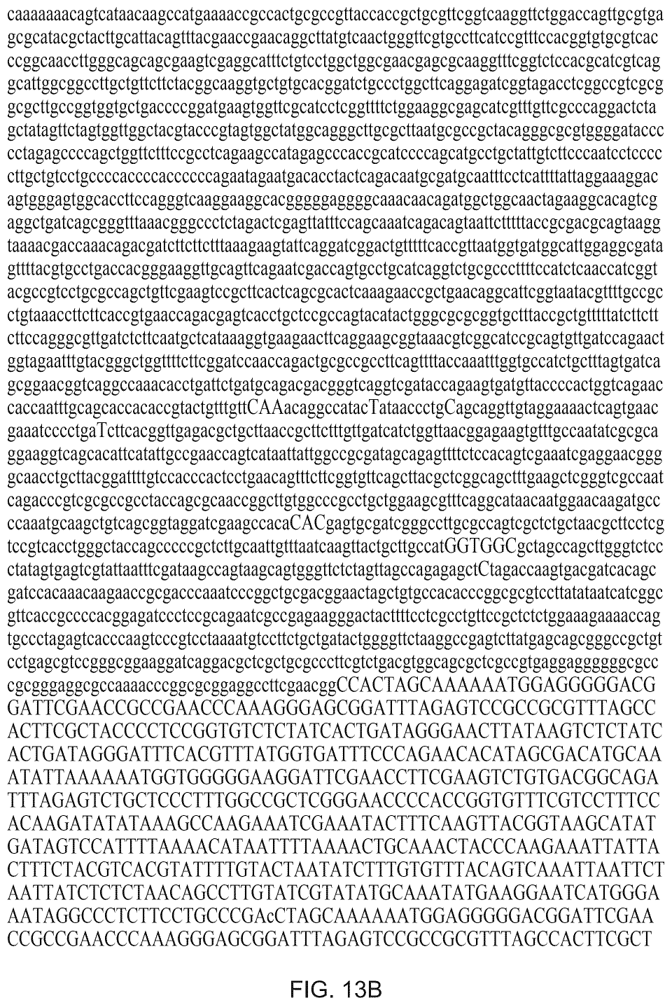

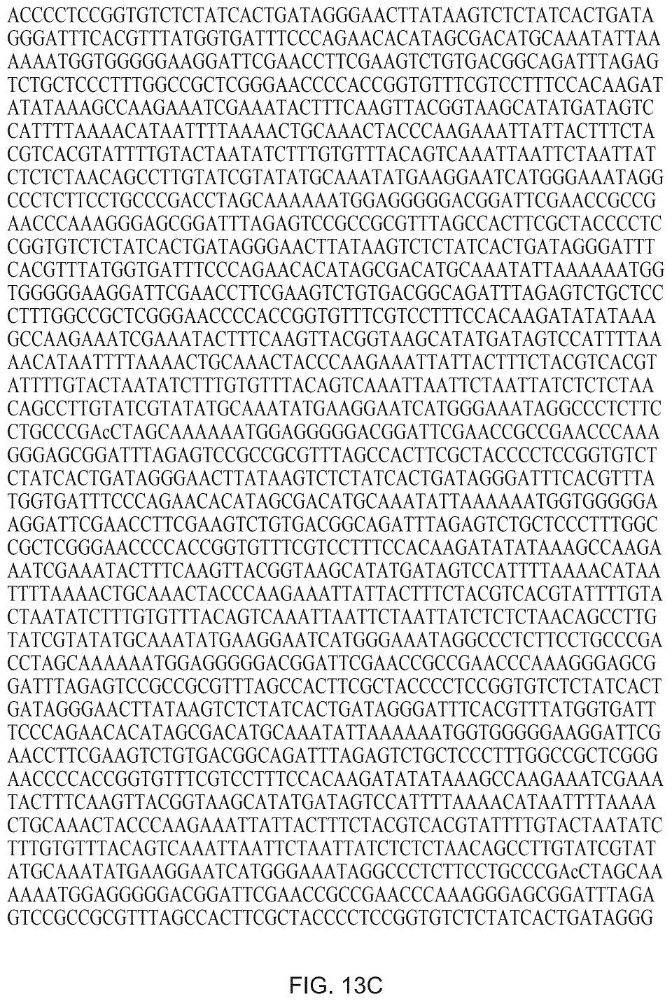

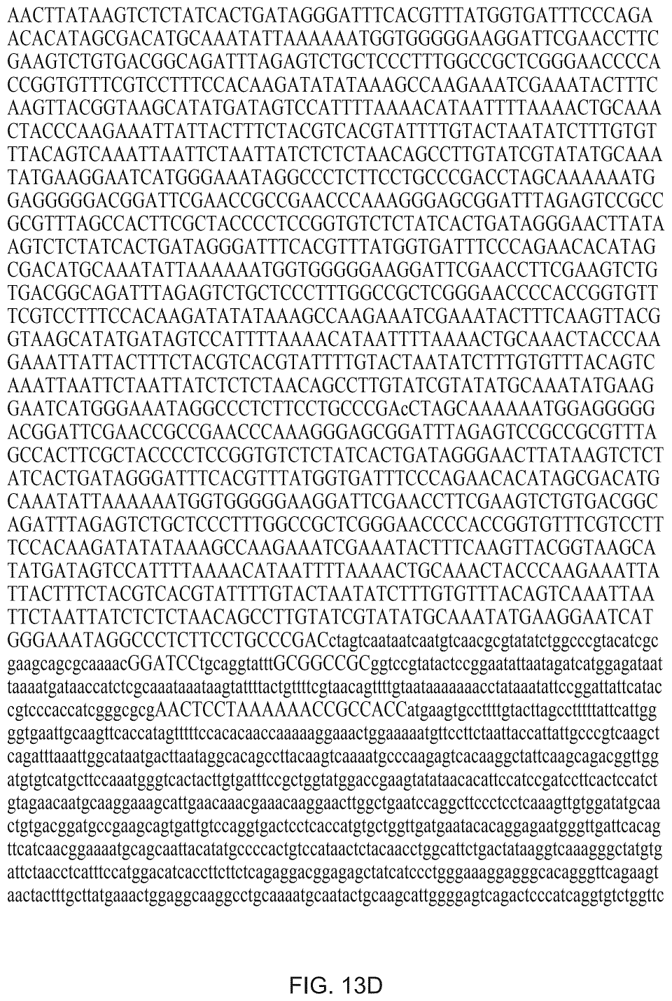

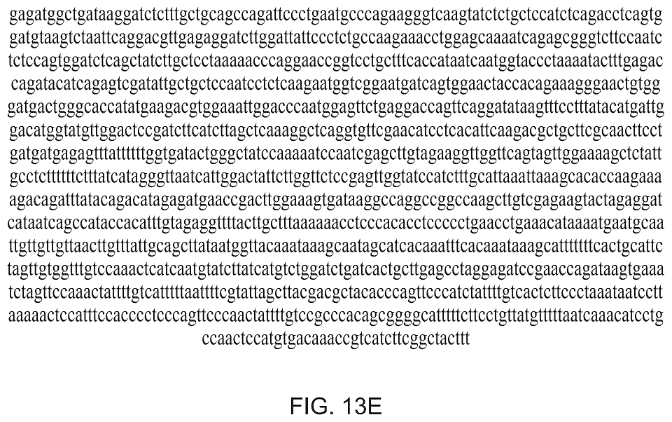

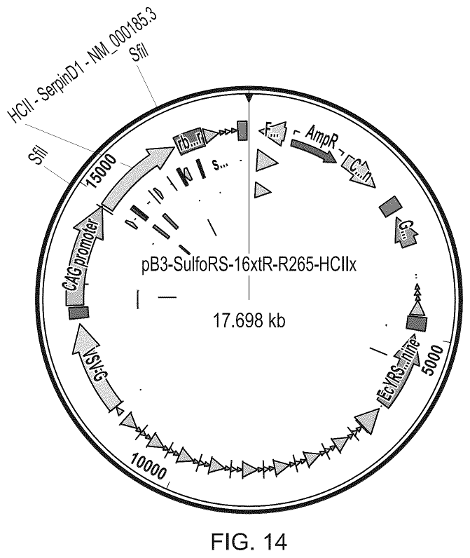

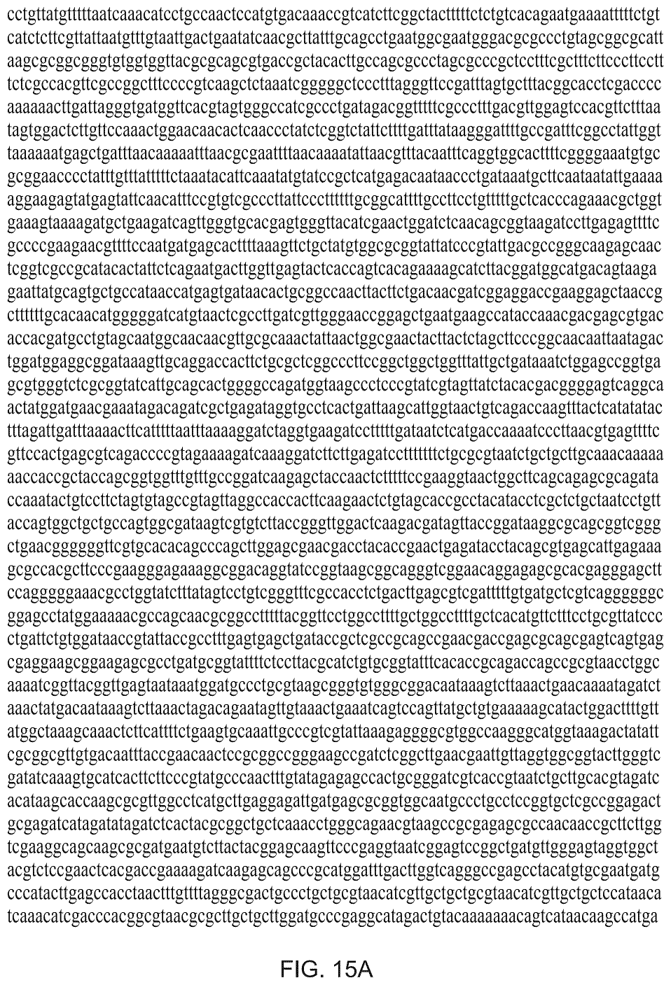

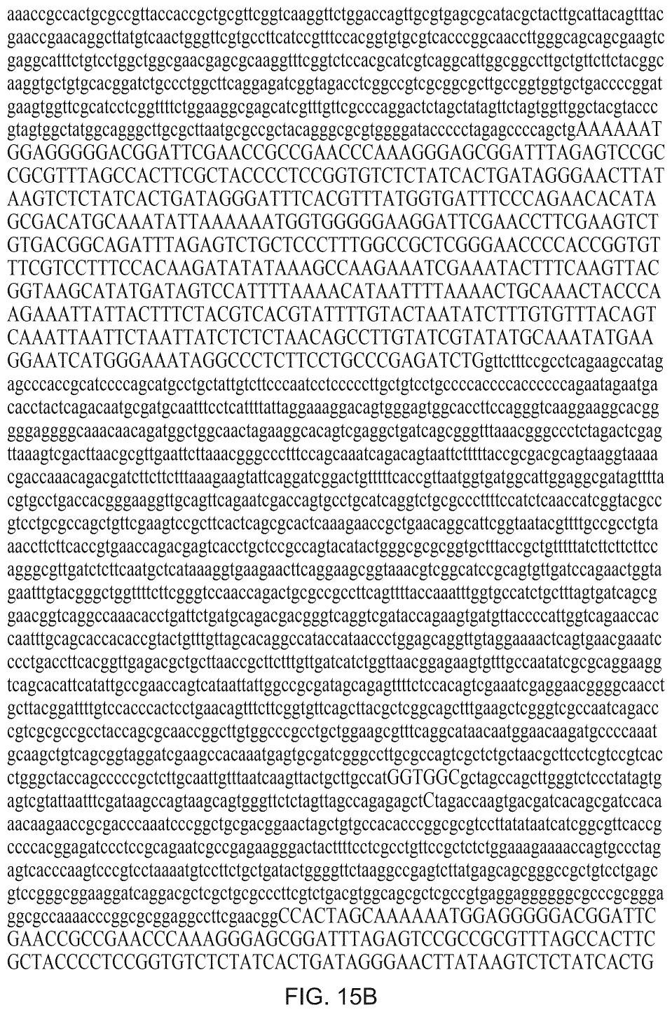

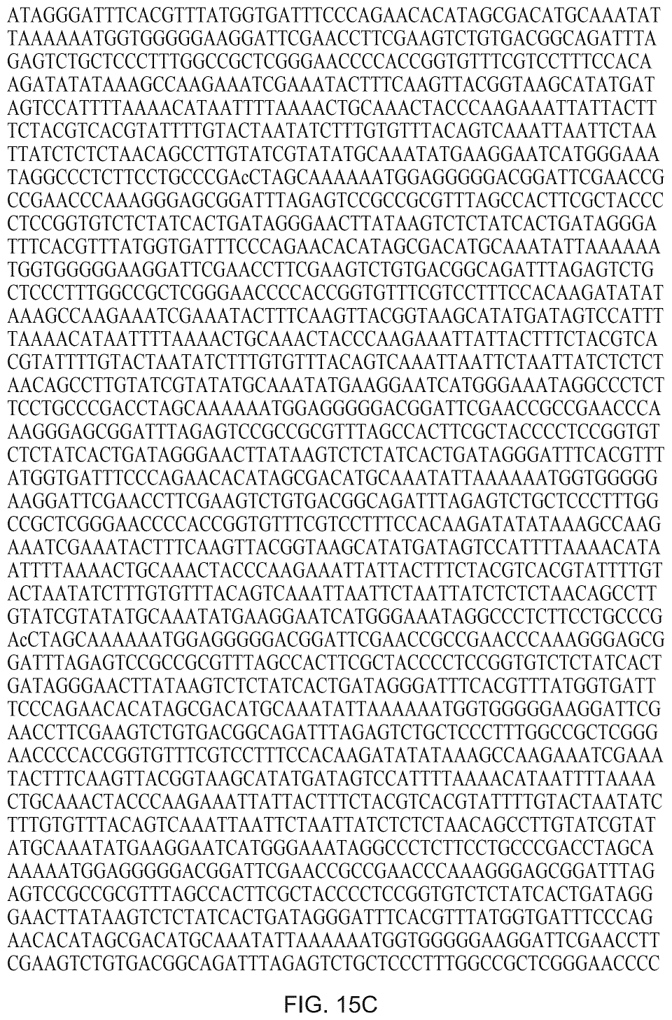

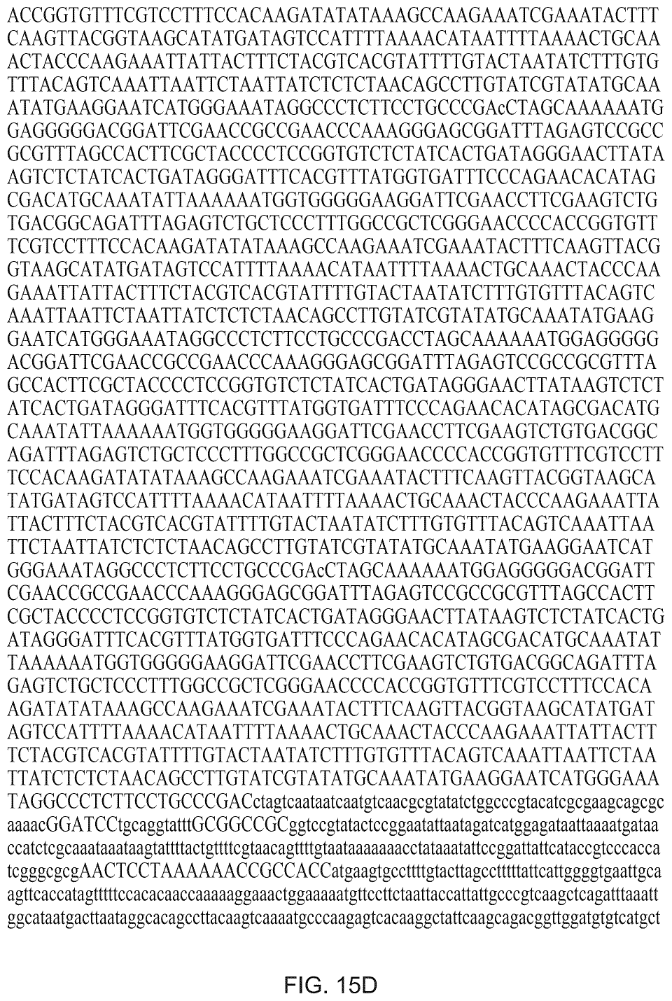

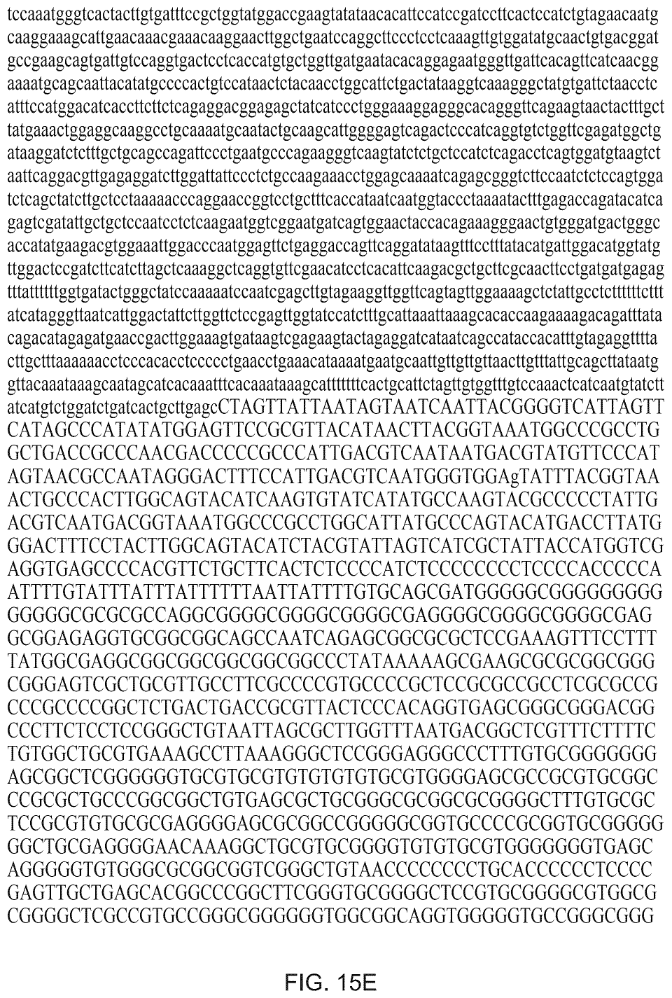

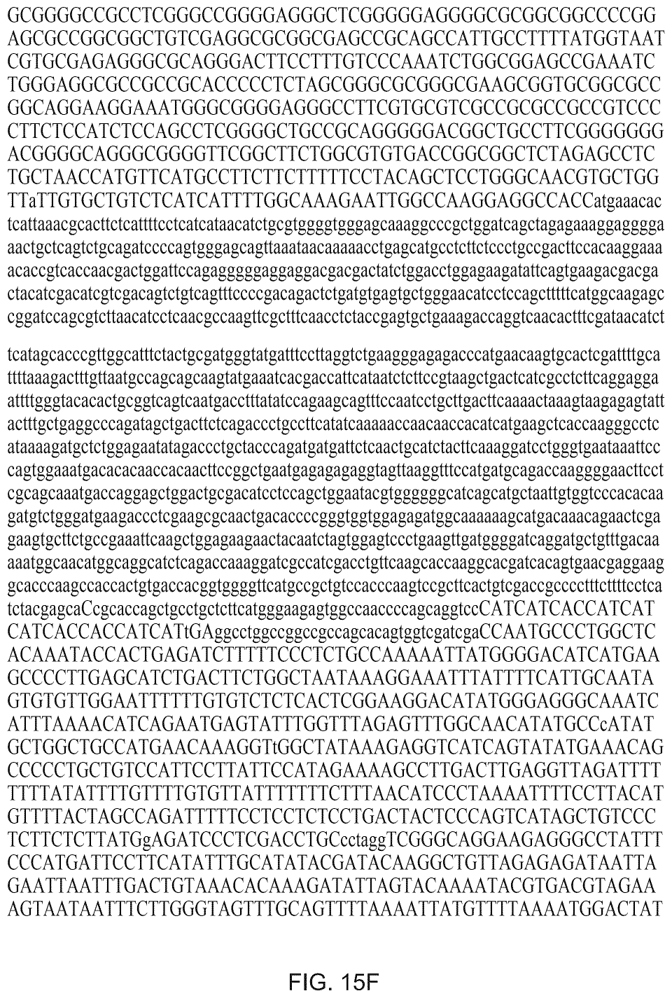

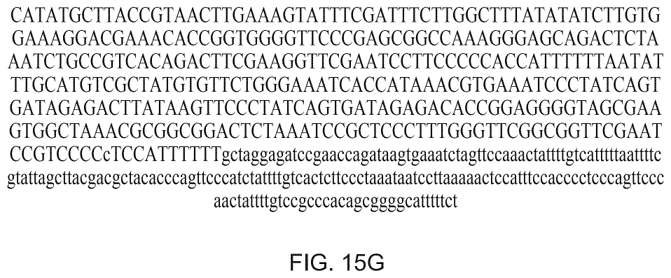

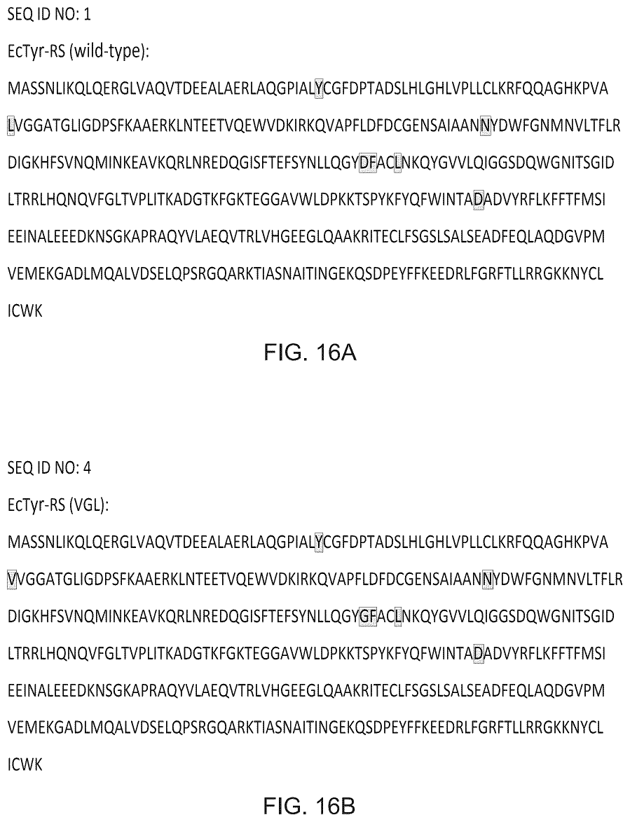

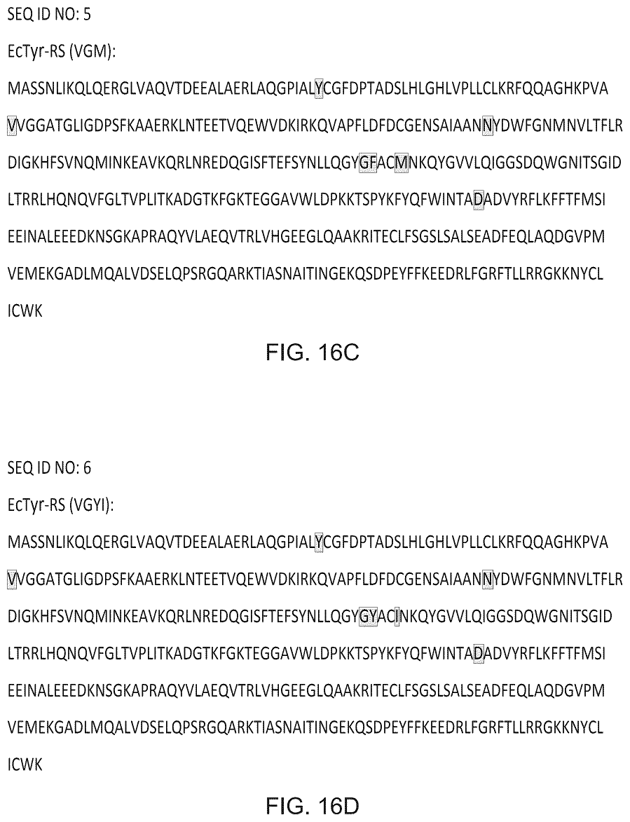

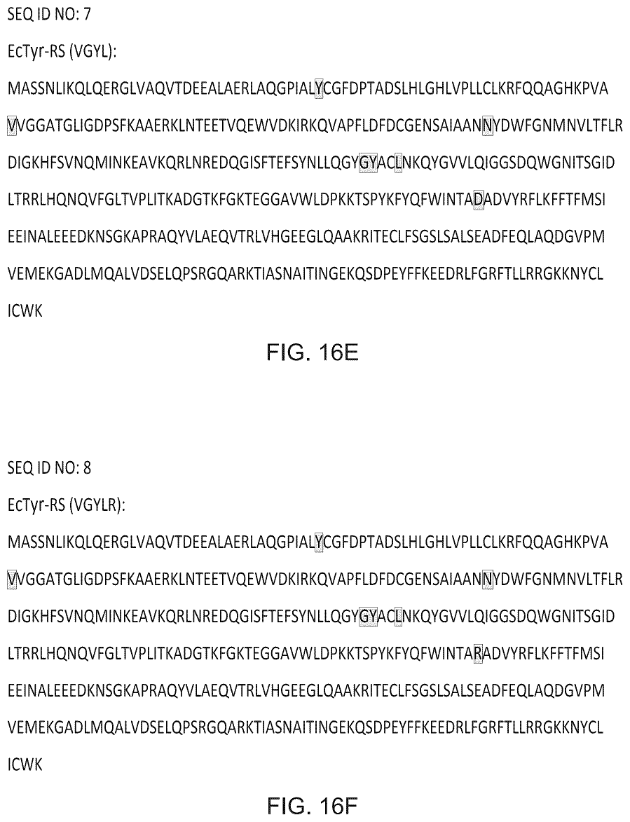

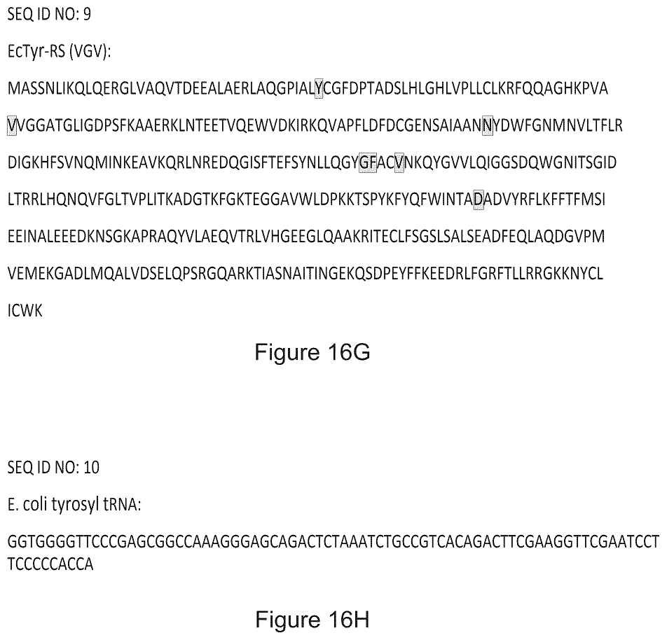

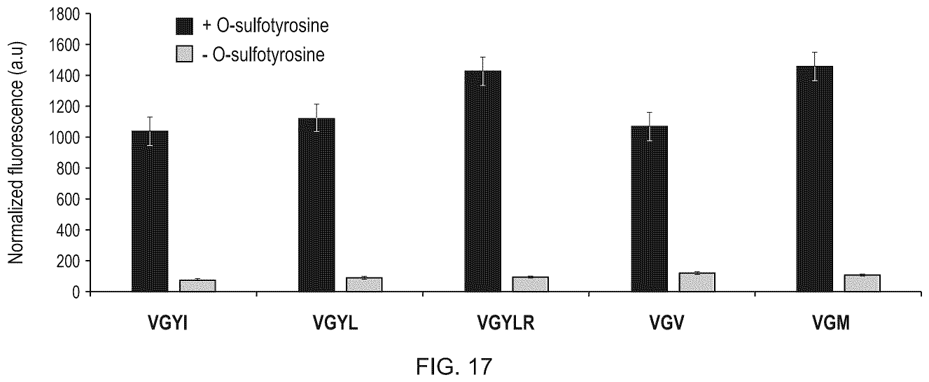

The platform of the present invention should allow facile expression of native eukaryotic proteins homogeneously sulfated at native sites. The present invention sought to demonstrate this using human heparin cofactor II (HCII) as a model system. HCII, a large secreted glycoprotein, is a serine protease inhibitor (serpin) that irreversibly inhibits thrombin, a key player in executing blood coagulation. This anticoagulant activity of HCII is triggered by glycosaminoglycans (GAGs) such as heparin. In the absence of GAGs, the acidic N-terminal domain (AND) of HCII binds its glycosoaminoglycan binding domain (GBD), resulting in an auto-inhibited state (FIG. 3a). GAGs activate HCII by binding its GBD and displacing the AND, which then recruits thrombin by binding its exosite 1 (FIG. 3a). The AND of HCII, which can bind both thrombin exosite I and GBD, is sulfated at two distinct sites (Tyr60 and Tyr73) whose roles in HCII activity is poorly understood. The absence of ER-Golgi processing precludes bacterial expression of HCII in its native glycosylated state, while overexpression in eukaryotic hosts can result in incomplete sulfation. UAG codons were introduced at 60 and 73 positions of full-length human HCII and overexpressed it in HEK293T cells in the presence of our sTyr incorporation system. Full-length HCII was successfully isolated from the culture medium using a C-terminal polyhistidine tag (FIG. 8). Whole-protein ESI-MS of this large protein was challenging, but the presence of sTyr at both sites through protease digestion followed by LC-MS analysis (FIG. 9, 10) was confirmed. Glycosylase (PNGase) treatment significantly reduced the molecular weight of the protein (FIG. 11), suggesting the presence of robust N-linked glycosylation. These results confirm that the platform of the present invention can be used to express endogenous eukaryotic proteins precisely sulfated at multiple sites.

In addition to 60-sTyr-73-sTyr, we also expressed and purified HCII mutants 60-Phe-73-Phe (to prevent sulfation), 60-sTyr-73-Phe, and 60-Phe-73-sTyr (FIG. 8) and evaluated their thrombin inhibition activities using an established biochemical assay. For each HCII mutant, second-order rate constants (k2) of thrombin inhibition were measured at different heparin concentrations to find the optimal [heparin], at which maximal inhibition rate is observed (FIG. 3b). 60-sTyr-73-sTyr exhibited a maximal rate constant of 3×10⁸ M⁻¹ min⁻¹ at ˜20 μg/mL heparin, which is in close agreement with previously reported data. Interestingly, the absence of sTyr at site 73 (60-sTyr-73-Phe) led to a slightly lower maximal k₂ but a substantially reduced (˜3 fold) optimal [heparin], whereas the 60-Phe-73-sTyr mutant (no sTyr at site 60) had an unchanged optimal [heparin] but a significantly lower maximal k₂ (FIG. 3b). The 60-Phe-73-Phe mutant showed both a low maximal k₂, and a reduced optimal [heparin]. The preliminary biochemical evaluation of precisely sulfated HCII mutants suggests important-yet distinct-roles the two sulfation PTMs play in fine-tuning its GAG-triggered thrombin inhibition activity: while the 73-sTyr appears to contribute more to AND-GBD association, the 60-sTyr might be more important for thrombin recruitment.

Without further elaboration, it is believed that one skilled in the art can, based on the above description, utilize the present invention to its fullest extent. The following specific embodiments and examples are, therefore, to be construed as merely illustrative, and not limitative of the remainder of the disclosure in any way whatsoever

### EXAMPLES

### Materials and Methods

### General Biological Reagents, Strains, and Protocols

Using E. coli strain DH10B (Life Technologies) was used for plasmid propagation and cloning. E. coli strains were cultured on LB-agar plates with appropriate antibiotic concentrations as follows: 95 μg/mL spectinomycin, 50 μg/mL chloramphenicol, 30 μg/mL kanamycin. Phusion high fidelity DNA polymerase (Thermo-Fischer) was used for PCR amplifications and restriction enzymes were obtained from New England Biolabs. DNA oligonucleotides were purchased from Integrated DNA Technologies, while Sanger sequencing was performed by Eton Bio. Engineered E. coli strain ATMY3 (contains one genomic copy of tRNA^{EcTyr}_{CUA}; no genomic EcTyrRS) was used as the selection host for the directed evolution of EcTyrRS. Engineered E. coli strain ATMY4 (contains two genomic copies of tRNA^{EcTyr}_{CUA}; no genomic EcTyrRS) was used as the expression host for expressing recombinant proteins incorporating sTyr.

HEK293T cell line was purchased from ATCC (ATCC CRL-3216) and maintained in DMEM (high glucose) supplemented with 10% FBS and Penicillin/Streptomycin. Cells were grown in a 37° C. 100% humidity, 5% CO₂.

### EcTyrRS Library Construction

In the EcYRS1 library (pBK-EcYRS1), six residues were randomized as follows: Y37-FLIMVSTAYHCG (SEQ ID NO:2), L71-NBT, N126-NSPTACGDH (SEQ ID NO: 3) D182-NST, F183-NNK, L186-NNK. A previously reported library (pBK-EcYRS1a) was used, which contains the desired Y37, D182, F183, and L186 randomizations, as the template to generate pBK-EcYRS1 by sequential overlap of extension PCR. Piece A was amplified with primers pBK seqT-F and EcYRS-L71-oR. Piece B was amplified with EcYRS-L71-NBT-F and EcYRS-N126-oR, and subsequently overlapped with piece A using terminal primers pBK seqT-F and EcYRS-N126-oR to create piece AB. Lastly, piece C was amplified with EcYRS-N126x-F (x corresponds to nine different codons) and pBK MCS JIsqR for all desired N126 variants. Piece C variants were combined in equal distribution and were subsequently overlapped with piece AB to form the full length aaRS PCR product.

After amplification, the aaRS PCR product was digested with NdeI/NcoI (NEB) and ligated by T4 DNA Ligase (NEB) into the pBK vector digested with the same restriction enzymes. The ligation mixture was ethanol precipitated with Yeast-tRNA (Ambion) and transformed into electrocompetent DH10B cells. Greater than 10⁸ transformants were obtained to ensure library coverage.

### Directed Evolution of EcTyrRS-SulfoY Variant in ATMY3

Positive selection 1: The pBK-EcYRS1 library was transformed into ATMY3 containing the positive selection plasmid pRepTrip2.3P-EcQtR-2x. The pRep plasmid expresses a chloramphenicol acetyl transferase (CAT) reporter containing a Q98TAG, an ampicillin resistance gene containing a 3TAG, an arabinose inducible T7 RNA polyermase containing two TAG codons (site 8 and 14), a T7 promoted GFPuv, and two copies of the E. coli tRNA^{Gln} expressed from its endogenous promoter. Approximately 9×10⁸ colony forming units were plated on LB+0.5× spectinomycin, tetracyclin, and kanamycin+0.02% arabinose+30 μg/mL ampicillin+30 or 50 μg/mL chloramphenicol in the presence of 1 mM sTyr for 18 h at 37° C. After 18 h, colonies from plates were harvested with 15 mL LB, centrifuged and selected pBK plasmid pool (pBK-EcYRS1a-P1) was purified via miniprep and isolated via gel purification.

For Negative selection: The isolated plasmid was subsequently transformed into ATMY3 containing pNeg2-2xQtR (contains arabinose dependent barnase with 3TAG, 45TAG, and two copies of the E. coli tRNA^{Gln}). Approximately 10⁸ cells were plated on LB-agar plates containing 0.5× spectinomycin, ampicillin, and kanamycin+0.02% arabinose in the absence of sTyr for 12 h at 37° C. After 12 h, colonies from plates were harvested with 15 mL LB, centrifuged and the pBK library subjected to one positive selection and one negative selection (pBK-EcYRS1a-P1N1) was purified via miniprep.

Positive selection 2: pBK-EcYRS1a-P2N1 was subjected to a second round of positive selection (10⁶ cfu plated). 96 single colonies from the second round of positive selection plates containing 1 mM sTyr were picked into 500 μL LB supplemented with spectinomycin, tetracyclin, kanamycin in a 96 deep-well plate and grown to confluence overnight. These overnight cultures were diluted 100 fold and 3 μL were individually spot plated on LB-Agar plates containing spectinomycin, tetracyclin, kanamycin+0.02% arabinose and 30 or 50 μg/mL chloramphenicol in the presence or absence of 1 mM sTyr. Eight pBK variants showing the most sTyr-dependent survival were picked for further characterization.

Characterization of tRNA/aaRS Activity in E. coli Via sfGFP Reporter

Preparation ATMY4 containing pEvolT5-sfGFP151TAG was transformed with pBK-EcTyrRS variants. Overnight starter cultures were diluted 100 fold in 10 mL LB containing required antibiotics and grown at 37° C. while shaking at 250 rpm in 50 mL flasks. Upon reaching 0.55 OD₆₀₀, 1 mM final IPTG was added to induce protein expression. 1 mL aliquots of induced cultures were placed in 15 mL culture tubes with or without 1 mM sulfotyrosine and grown for 18-20 h at 30° C. Afterwards, cells were pelleted, resuspended in PBS, and diluted 10 fold. Dilutions were transferred to a 96-well clear bottom plate. Expression of full-length sfGFP was measured using the associated characteristic fluorescence by a SpectraMAX M5 (Molecular Devices) multimode plate reader (Ex. 488 nm; Em. 534 nm; 515 cutoff) and normalized with respect to OD₆₀₀.

Purification of sfGFP-TAG from Bacterial Expression

Protein expression was performed in 10 mL culture as described above (sfGFP151-TAG reporter assay). Afterwards, the cells were pelleted at 5000×g, resuspended in lysis buffer [B-PER Bacterial Protein Extraction Reagent (Thermo Scientific), 1× Halt Protease Inhibitor Cocktail (Thermo Scientific), 0.01% Pierce Universal Nuclease (Thermo Scientific), and incubated for 10 min on ice. After incubation, the crude lysate was clarified at 22,000×g. The full-length sfGFP containing a C-terminal 6×HisTag (SEQ ID NO: 33) was purified using HisPur Ni-NTA resin (Thermo Scientific) according to the manufacturers protocol. SDS-PAGE and Bradford analysis were used to assess protein purity, while the molecular weight was confirmed by ESI-MS (Agilent Technologies 1260 Infinity ESI-TOF).

Site-Specific Incorporation of sTyr into Protein Expressed in Mammalian Cells

HEK293T cells were maintained as described above. pBIU-SulfoA1-16xtYR-TAG (VGL) or pB1U-SulfoB7-16xtYR-TAG (VGM) contain 16 copies of alternating U6/H1 promoted E. coli tRNA^{Tyr}_{CUA} and UbiC promoted EcTyrRS mutants. pAcBac1-EGFP-39TAG was used as a reporter plasmid. 0.7×10⁶ cells per well were seeded one day prior to transfection in a 12 well plate. At 70% confluence, the transfection mixture (500 ng each of suppressor and reporter plasmid, 18 μL DMEM, 3.5 μL Sigma PEI (1 mg/mL), 10 min incubation prior to addition) was added to each well and gently mixed. A final concentration of 2 mM sTyr was added to the wells at the time of transfection. After 48 h, cells were harvested by centrifugation at 5000×g and residual media was removed. 50 μL lysis buffer (10 mL CellLytic M, 1× Halt Protease inhibitor, 0.01% Pierce universal nuclease) was added per well and incubated for 10 min. After incubation, cells were clarified by centrifugation and lysate was analyzed for fluorescence in the SpectraMAX M5 (Molecular Devices) under the same conditions as sfGFP.

For purification and further characterization, EGFP-39-sTyr was expressed in 10 cm dishes (8.5×10⁶ seeded 24 h prior to transfection). 5 μg suppressor plasmid and 5 μg reporter plasmid were incubated with 180 μL DMEM (no FBS) and 40 μL PEImax (Polysciences; 1 mg/mL). 2 mM sTyr and 2 mM Sodium Butyrate was added at the time of transfection. After 48 hr, cells were harvested at 5000×g. 600 μL lysis buffer (CellLytic M, 1× Halt protease inhibitor, 0.01% Pierce universal nuclease) was used to lyse the cells. After 10 min incubation, the lysate was clarified by centrifugation and the protein was purified using HisPur Ni-NTA resin (Thermo-Scientific). Purity and the molecular weight of the expressed protein was analyzed by SDS-PAGE and ESI-MS (Agilent Technologies 1260 Inifinity ESI-TOF).

### Anti-his and Anti-Sulfotyrosine Western Blot of GFP Reporters

Western blot was used to confirm the presence of a polyhistidine tag (via anti-HisTag blot) and the presence of sulfotyrosine (via anti-sTyr blot) in reporter proteins expressed above. 500 ng each of purified wild-type or sTyr-incorporated mutant of sfGFP or EGFP reporter proteins were resolved by SDS-PAGE, and transferred to a PVDF membrane (Life Technologies) using a Trans-Blot Turbo Transfer System (BioRad) in Towbin Transfer Buffer (at 12V for 30 min, twice). After complete transfer, membrane was blocked in 10 mL 5% milk in TBST (HisBlot) or 10 mL Pierce Superblocker (Fisher Scientific) at 4° C. overnight with constant agitation. Membranes were subsequently incubated in 1:3000 anti-HisTag mouse mAb (Invitrogen, MA121315, in 5% milk TBST) or 1:6000 anti-Sulfotyrosine mouse mAb (Millipore Sigma, Clone: 1CA2, in Pierce Superblocker) overnight. Next, the membrane was washed six times, 10 min per wash, using TBST at room temperature. Afterwards, 1:6000 dilution of chicken anti-mouse secondary antibody (Invitrogen, SA1-72021, in 5% milk TBST) was incubated for 2 h at room temperature. The membrane was washed and activated using SuperSignal West Dura Kit (Fisher Scientific). The activated blot was imaged on the ChemiDoc MP imaging system (BioRad).

### Expression and Purification of Heparin Cofactor II (HCII)

HEK293T cells were maintained as described above. pB3-SulfoRS-16xYtR-TAG-HCII contains the following three components: 16 copies of alternatingly H1/U6 promoted E. coli tyrosine tRNA_{CUA}, a UbiC promoted EcTyrRS mutant, and HCII mutants under a CAG promoter. 10 cm dishes were seeded with 8.5×10⁶ cells 24 h prior to transfection. Afterwards, DMEM+FBS media was aspirated and replaced with DMEM without FBS. A transfection mixture (10 μg pB3 plasmid, 180 μL DMEM, 50 μL PEI Max) was incubated for 10 min prior to addition. 2 mM sTyr and 2 mM sodium butyrate were added at the time of transfection. Since HCII is a secreted protein, the media was harvested on days 2 and 3 post transfection, stored at 4° C. for up to 2 days, and adherent HCII expressing cells were re-supplemented with DMEM (no FBS)+2 mM sTyr+2 mM sodium butyrate. Collected media containing overexpressed HCII (20 mL total per 10 cm plate) were pooled and subjected to purification.

HCII containing media was centrifuged at 5,000×g at 4° C. for 30 min to remove any residual debris. The supernatant was concentrated with Amicon 30 kDa MWCO centrifugal filters to approximately 2 mL. For concentrated media harvested from five 10 cm dishes, 1 mL Ni-NTA (Thermo-Scientific) resin was used for protein purification. Bound protein was washed with 50 mL of wash buffer containing PBS+45 mM imidazole. HCII was eluted with 10 mL elution buffer, concentrated down to 1 mL using a 30 kDa MWCO filter, and buffer exchanged into HNPN-PEG buffer (20 mM HEPES, pH 7.4, 150 mM NaCl, 0.05% NaN₃). Protein yields and purity were analyzed by Bradford, SDS-PAGE, anti-His tag dot blot, and tryptic/elastase mass spectrometry.

### Deglycosylation Assay of HCII

PNGase F was purchased from Promega (V4831). 18 μL (10 μg, in 0.5 mM Tris-HCl, pH 7.8) purified recombinant HCII was incubated at 37° C. with or without 2 μL PNGase for 18 hrs. After incubation, mixtures were resolved by SDS-PAGE and imaged via ChemiDoc imaging.

### Tryptic & Elastase Mass Spectrometry Characterization of HCII

An in gel digestion was performed to prepare peptides for MS analysis. 1000-2000 ng HCII was resolved by SDS-PAGE. Gel was stained for 1 hr, and destained overnight. After destain, HCII bands were sliced and cut into approximately 1 mm² pieces. Pieces were placed in microcentrifuge tubes containing 500 μL 100 mM ammonium bicarbonate. Gel bands were frozen at −80° C. overnight in the 500 μL ammonium bicarbonate. Gel bands were thawed, supernatant was removed, and gel bands were washed 1-2× for 15 min with 500 μL 100 mM ammonium bicarbonate. After washes, supernatant was removed and 200 μL 10 mM TCEP was added to completely cover gel bands. Samples were placed in a 60° C. water bath for 30 min. Samples were quickly spun and TCEP was aspirated. 200 μL 55 mM iodoacetamide was added to cover the gel bands. Tubes were placed in the dark for 30 min at RT. Supernatant was removed and gel bands were washed 3× for 15 min in 500 μL 50:50 acetonitrile: 100 mM ammonium bicarbonate. After washes, supernatant was removed and 50 μL acetonitrile was added to completely dehydrate the gel bands (turned opaque). Acetonitrile was removed and residual solvent was removed using a SpeedVac for 5 min.

Sequencing grade trypsin (V5111) and neutrophil elastase (V1891) was purchased from Promega. Sample was resuspended in either trypsin (for 60 site) or trypsin+elastase (for 73 site). For trypsin, 200 ng trypsin (20 μL, resuspended in 25 mM ammonium bicarbonate) was added to dehydrated gel slices. For trypsin+elastase, 300 ng (30 μL, 25 mM ammonium bicarbonate) trypsin was added, immediately followed by 35 μL elastase (30 ng, resuspended in double distilled water according to manufacturer protocol)+50 μL 50 mM Tris-HCl. In both cases, enzymes were incubated with gel sample for 10 min before 200 μL 50 mM ammonium bicarbonate was added and placed at 37° C. incubator overnight. Next, the supernatant was transferred to a clean tube and 100 μL formic acid was added to the gel bands followed by a 15 min incubation at RT. The supernatant was aspirated and combined with the supernatant from the last step. Formic acid washes of the gel slices were repeated two more times. Next, 150 μL acetonitrile was added to cover the gel slices, incubated at RT for 15 min, and combined with all previous washes. Acetonitrile washes were repeated two more times until bands became opaque. Lastly, the peptide sample (˜500 μL consisting of the overnight incubation supernatant, formic acid washes, and acetonitrile washes) was evaporated down to 10 μL using SpeedVac and stored at −80° C. until subjected to HPLC-MS analysis.

Digested peptides were analyzed by LC-MS using an LTQ Orbitrap XL mass spectrometer (Thermo Fisher) coupled to an EASY-nLC 1000 nanoLC (Thermo Fisher). 18 μL of sample was loaded onto 100 μm fused silica column with a 5 μm tip packed with 10 cm of Aqua C18 reverse-phase resin (Phenomenex) using the EASY-nLC 1000 autosampler. Peptides were eluted with a gradient 0-55% buffer B in buffer A (buffer A: 95% water, 5% acetonitrile, 0.1% formic acid; buffer B; 20% water, 80% acetonitrile, 0.1% formic acid). The flow rate through the column was set to 400 nl/min and the spray voltage was set to 3.5 kV. One full MS scan (400-1800 MW) was followed by seven data dependent scans. For the data dependent scans, a mass list was used to target the predicted peptides with sTyr at residues 60 and 73. In the absence of a targeted peptide, data dependent scans were performed on the nth most intense ions in the MS1. MS1 spectra and total ion chromatograms were manually analyzed for peptide identification and presence of sulfation at each residue.

### HCII-Thrombin Activity Assay

To calculate the second order rate constant of thrombin inhibition by HCII, thrombin was incubated with excess HCII under pseudo-first order conditions in the presence of different heparin concentrations (details below). The reaction was quenched after 1 minute and the residual thrombin activity (k_{inhibited}) was measured using a chromogenic substrate. The pseudo-first order rate constant (k₁) was calculated from this using the equation k₁=−ln(k_{inhib}/k_{uninhib})/t, where k_{uninhib} is the activity of thrombin in the absence of HCII inhibition under identical treatment. The second order inhibition rate constant (k₂) was calculated from k₁ using the equation k₂=k₁/[HCII] with units of M⁻¹ min⁻¹. The second order rate constant at each heparin concentration was plotted against the corresponding heparin concentration.

Concentrations of different HCII protein were measured by Bradford and normalized by anti-His dot-blot assay (blot intensities quantified via ChemiDoc imaging). Clear plastic 96 well plates were coated with 2 mg/mL ovalbumin (Fisher) for 1 hr at 37° C. Ovalbumin was removed by tapping the plate on a paper towel. A master mix of 2 mg/mL ovalbumin, 0-2 mg/mL heparin (Fisher), 0.6 nM α-thrombin (Fisher) in HNPN (20 mM HEPES, pH 7.4, 150 mM NaCl, 0.05% NaN3) were incubated in the treated 96 wells for 1 min with 10 nM HCII. After 1 min, 10 μL of a solution of 1 mg/mL polybrene was directly added to all wells using a multichannel pipet to quench the heparin-dependent inhibition of thrombin by HCII. The plates were spun down in a bucket centrifuge for 10 min at 3,500 rpm to precipitate the heparin/polybrene complex. 100 μL supernatant was removed and 50 μL 450 μM ChromozymeTH (Sigma) substrate was added to measure the amount of residual thrombin activity by monitoring the absorbance on the SpectroMax plate reader at 405 nm for 1 hr in triplicate.

### List of Oligonucleotides Shown Below in Table 1 (SEQ ID NOS: 17-32)

| | Primer Name | Sequence |
|---|---|---|
| SEQ ID | pBK seqT-F | ATTACGCTGACTTGACGGGACGG |
| NO: 17 | | |
| SEQ ID | EcYRS-L71-oR | CGCAACCGGCTTGTGGCCCGCCTGC |
| NO: 18 | | |
| SEQ ID | EcYRS-L71-NBT-F | GCAGGCGGGCCACAAGCCGGTTGCG |
| NO: 19 | | nbtGTAGGCGGCGCGACGGGTCTGA |
| | | TTG |
| SEQ ID | EcYRS-N126-oR | GTTCGCCGCGATAGCAGAGTTTTC |
| NO: 20 | | |
| SEQ ID | EcYRS-N126x-F | GAAAACTCTGCTATCGCGGCGAACn |
| NO: 21 | | nnTATGACTGGTTCGGCAATATGAA |
| | | TGTGCTGAC |
| SEQ ID | pBK MCS JIsqR | GAGATCATGTAGGCCTGATAAGCGT |
| NO: 22 | | AGC |
| SEQ ID | EcYRS-NheI-F | GCTAGCGCCACCATGGCAAGCA |
| NO: 23 | | |
| SEQ ID | EcYRS-XhoI-R | aataatCTCGAGTTATTTCCAGCAA |
| NO: 24 | | ATCAGACAGTAATTCTTTTTACC |
| SEQ ID | HCII-SfiI-F | TGGCAAAGAATTGGCCAAGGAGGCC |
| NO: 25 | | ACCATGAAACACTCATTAAACGCAC |
| | | TTC |
| SEQ ID | 10xH is-TGA-SfiI-R | TGGCGGCCGGCCAGGCCTCAATGAT |
| NO: 26 | (“1.0xHis” | GGTGGTGATGATGATGGTGATGATG |
| | disclosed as SEQ | |
| | ID NO: 34) | |
| SEQ ID | HCII-79-Phe-R | GTCGTCGTCTTCACTGAATATCTTC |
| NO: 27 | | TCCAGGTCCAGaaaGTCGTCGTCCT |
| | | CCTCCCCC |
| SEQ ID | HCII-79-TAG-R | GTCGTCGTCTTCACTGAATATCTTC |
| NO: 28 | | TCCAGGTCCAGctaGTCGTCGTCCT |
| | | CCTCCCCC |
| SEQ ID | HCII-92-Phe-F | CTGGACCTGGAGAAGATATTCAGTG |
| NO: 29 | | AAGACGACGACtttATCGACATCGT |
| | | CGACAGTCTG |
| SEQ ID | HCII-92-TAG-F | CTGGACCTGGAGAAGATATTCAGTG |
| NO: 30 | | AAGACGACGACtagATCGACATCGT |
| | | CGACAGTCTG |
| SEQ ID | HCII-80-iF | CTGGACCTGGAGAAGATATTCAGTG |
| NO: 31 | | AAGACGACGAC |
| SEQ ID | HCII-80-iR | GTCGTCGTCTTCACTGAATATCTTC |
| NO: 32 | | TCCAGGTCCAG |

### Plasmid Content and Construction

pB1U-Sulfo-16xtYR-TAG: EcTyrRS VGL and VGM variants were amplified from pBK with oligonucleotides EcYRS-NheI-F and ExYRS-XhoI-R and subcloned into pB1U-OMe YRS-16xtYR-TAG between NheI and XhoI.

pB3-SulfoRS-16xYtR-TAG-HCII: pAcBac3 OMeYRS was used as a starting vector to construct this plasmid. pB3 (abbreviated pAcBac3) is identical to pBlu except it contains a CAG promoter upstream from an SfiI site as well as 4 additional tRNA cassette copies. OMeYRS was replaced with SulfoRS via NheI/XhoI as previously described in pB1U cloning description. The SfiI site was used to insert HCII. HCII-SfiI-F and 10xHis-TGA-SfiI-R (“10xHis” disclosed as SEQ ID NO: 34) were used to amplify HCII from pCMV-SerpinD1 (Origine, SC120039). Mutations were introduced via overlap extension (see primer list for 79, 92, and 80 overlap primers-79 and 92 correspond to 60 and 73 sites, respectively). HCII insert and vector were digested with SfiI and ligated via traditional RE cloning.

In summary, the present invention has developed a platform for site-specific incorporation of sTyr into proteins expressed in eukaryotic cells with high fidelity and efficiency, which would be a valuable tool for investigating the consequences of tyrosine sulfations found in the eukaryotic proteome. This platform can also be used to express therapeutically relevant proteins homogeneously modified with functionally important sulfations. Additionally, the ability to incorporate sTyr into virtually any site of any protein in eukaryotic cells offers intriguing opportunities for novel synthetic biology applications.

### REFERENCES

The references listed in this application are herein incorporated by reference in their entirety.
1 Moore, K. L. Protein tyrosine sulfation: a critical posttranslation modification in plants and animals. Proceedings of the National Academy of Sciences of the United States of America 106, 14741-14742, doi:10.1073/pnas. 0908376106 (2009).
2 Seibert, C. & Sakmar, T. P. Toward a framework for sulfoproteomics: Synthesis and characterization of sulfotyrosine-containing peptides. Biopolymers 90, 459-477, doi:10.1002/bip.20821 (2008).
3 Stone, M. J., Chuang, S., Hou, X., Shoham, M. & Zhu, J. Z. Tyrosine sulfation: an increasingly recognised post-translational modification of secreted proteins. New biotechnology 25, 299-317 (2009).
4 Yang, Y. S. et al. Tyrosine sulfation as a protein post-translational modification. Molecules (Basel, Switzerland) 20, 2138-2164, doi:10.3390/molecules20022138 (2015).
5 Farzan, M. et al. Tyrosine sulfation of the amino terminus of CCR5 facilitates HIV-1 entry. Cell 96, 667-676 (1999).
6 Huang, C.-c. et al. Structural basis of tyrosine sulfation and VH-gene usage in antibodies that recognize the HIV type 1 coreceptor-binding site on gp120. Proceedings of the National Academy of Sciences of the United States of America 101, 2706-2711 (2004).
7 Li, X., Hitomi, J. & Liu, C. C. Characterization of a Sulfated Anti-HIV Antibody Using an Expanded Genetic Code. Biochemistry (2018).
8 Stone, M. J. & Payne, R. J. Homogeneous sulfopeptides and sulfoproteins: synthetic approaches and applications to characterize the effects of tyrosine sulfation on biochemical function. Accounts of chemical research 48, 2251-2261 (2015).
9 Thompson, R. E. et al. Tyrosine sulfation modulates activity of tick-derived thrombin inhibitors. Nature chemistry 9, 909 (2017).
10 Mikkelsen, J., Thomsen, J. & Ezban, M. Heterogeneity in the tyrosine sulfation Chinese hamster ovary cell produced recombinant FVIII. Biochemistry 30, 1533-1537 (1991).
11 Chin, J. W. Expanding and reprogramming the genetic code. Nature 550, 53 (2017).
12 Italia, J. S. et al. Expanding the genetic code of mammalian cells. Biochemical Society transactions 45, 555-562, doi:10.1042/bst20160336 (2017).
13 Young, D. D. & Schultz, P. G. Playing with the molecules of life. ACS chemical biology (2018).
14 Liu, C. C., Brustad, E., Liu, W. & Schultz, P. G. Crystal structure of a biosynthetic sulfo-hirudin complexed to thrombin. Journal of the American Chemical Society 129, 10648-10649 (2007).
15 Liu, C. C. & Schultz, P. G. Recombinant expression of selectively sulfated proteins in Escherichia coli. Nature biotechnology 24, 1436-1440, doi:10.1038/nbt1254 (2006).
16 Watson, E. E. et al. Mosquito-Derived Anophelin Sulfoproteins Are Potent Antithrombotics. ACS central science 4, 468-476 (2018).
17 Italia, J. S., Latour, C., Wrobel, C. J. & Chatterjee, A. Resurrecting the bacterial tyrosyl-tRNA synthetase/tRNA pair for expanding the genetic code of both E. coli and eukaryotes. Cell chemical biology 25, 1304-1312. e1305 (2018).
18 Chin, J. W. et al. An expanded eukaryotic genetic code. Science (New York, N.Y.) 301, 964-967, doi:10.1126/science.1084772 (2003).
19 Dumas, A., Lercher, L., Spicer, C. D. & Davis, B. G. Designing logical codon reassignment-Expanding the chemistry in biology. Chemical Science 6, 50-69 (2015).
20 Italia, J. S. et al. An orthogonalized platform for genetic code expansion in both bacteria and eukaryotes. Nature chemical biology 13, 446-450, doi:10.1038/nchembio.2312 (2017).
21 Tollefsen, D. M. Heparin cofactor II. Advances in experimental medicine and biology 425, 35-44 (1997).
22 Tollefsen, D. M. Heparin cofactor II modulates the response to vascular injury. Arteriosclerosis, thrombosis, and vascular biology 27, 454-460 (2007).
23 Hortin, G., Tollefsen, D. & Strauss, A. W. Identification of two sites of sulfation of human heparin cofactor II. Journal of Biological Chemistry 261, 15827-15830 (1986).
24 Ciaccia, A. V., Monroe, D. M. & Church, F. C. Arginine 200 of Heparin Cofactor II Promotes Intramolecular Interactions of the Acidic Domain IMPLICATION FOR THROMBIN INHIBITION. Journal of Biological Chemistry 272, 14074-14079 (1997).
25 Mitchell, J. W. & Church, F. C. Aspartic acid residues 72 and 75 and tyrosine-sulfate 73 of heparin cofactor II promote intramolecular interactions during glycosaminoglycan binding and thrombin inhibition. Journal of Biological Chemistry 277, 19823-19830 (2002).
26 Zheng, Y., Lewis Jr, T. L., Igo, P., Polleux, F. & Chatterjee, A. Virus-enabled optimization and delivery of the genetic machinery for efficient unnatural amino acid mutagenesis in mammalian cells and tissues. ACS synthetic biology 6, 13-18 (2016).

While this invention has been particularly shown and described with references to preferred embodiments thereof, it will be understood by those skilled in the art that various changes in form and details may be made therein without departing from the scope of the invention encompassed by the appended claims.

What is claimed is:

1. A composition comprising a variant E. coli tyrosyl-tRNA synthetase (EcTyr-RS) wherein the variant EcTyr-RS preferentially aminoacylates an E. coli tyrosyl-tRNA (EctRNAtyr) with a sulfotyrosine analog over the naturally-occurring tyrosine amino acid, wherein the variant EcTyr-RS comprises the amino acid sequence of SEQ ID NO: 1, or an amino acid sequence with at least 90% sequence identity with the full-length SEQ ID NO: 1, wherein the EcTyr-RS variant is mutated, relative to SEQ ID NO: 1, such that the leucine (L) at position 71 is replaced with valine (V), the aspartic acid (D) at position 182 is replaced with G, the phenylalanine at position 183 is either conserved or mutated to Y, and the L at position 186 is either conserved, or is replaced with M, I, or V.

2. The composition of claim 1, further comprising an E. coli tyrosyl tRNA, wherein the tRNA polynucleotide sequence comprises SEQ ID NO: 10 wherein the tRNA has an anti-codon loop comprising a sequence that specifically binds to a selector sequence of an mRNA, wherein the selector sequence is an amber codon.

3. The composition of claim 1, wherein the EcTyr-RS variant comprises an amino acid sequence selected from the group consisting of: SEQ ID NOS: 4-9.

4. The composition of claim 1, wherein the tyrosine analog is sulfotyrosine.

5. A method of producing a protein in a cell with one, or more, tyrosyl analogs at specified positions in the protein, the method comprising,
a. culturing the cell in a culture medium under conditions suitable for growth, wherein the cell comprises a nucleic acid that encodes a protein with one, or more, amber selector codons at specific sites in the protein, wherein the cell comprises an Ec-tRNA^{try} that recognizes the
selector codon and wherein the cell further comprises a nucleotide sequence encoding a variant E. coli tyrosyl-tRNA synthetase of claim 1, and
b. contacting the cell culture medium with one, or more, tyrosyl analogs under conditions suitable for incorporation of the one, or more, tyrosyl analogs into the protein in response to the selector codon,
thereby producing the protein with one, or more tyrosyl analogs.

6. The method of claim 5, wherein the Ec-tRNA^{tyr} polynucleotide sequence comprises SEQ ID NO: 10, or a homologous bacteria-derived tRNA comprising at least about 80% sequence identity with SEQ ID NO: 10, wherein the tRNA has an anticodon loop comprising a sequence that specifically binds to a selector sequence of an mRNA, wherein the selector sequence is an amber codon.

7. The method of claim 5, wherein the tyrosyl analog is sulfotyrosine.

8. The method of claim 5, wherein the cell is an E. coli cell or a eukaryotic cell.

9. The method of claim 8, wherein the eukaryotic cell is a mammalian cell.

10. The method of claim 8, wherein the E. coli cell is the ATMY4 strain of E. coli cell.

11. The method of claim 5, wherein the cell further comprises a second tRNA/RS pair that is orthogonal to the cell, wherein the second pair does not cross-react with the EcTyr-RS/tRNA pair and that recognizes an amber selector codon in the protein, wherein the protein produced contains one, or more tyrosyl analogs and one, or more, distinct unnatural amino acid other than a tyrosyl analog.

12. A method of site-specifically incorporating one, or more, suflotyrosine residues into a protein or peptide in a cell, the method comprising,
a. culturing the cell in a culture medium under conditions suitable for growth, wherein the cell comprises a nucleic acid that encodes a protein or peptide of interest with one, or more, amber selector codons at specific sites in the protein or peptide,
wherein the cell further comprises a variant E. coli tyrosyl-tRNA synthetase (EcTyr-RS), wherein the EcTry-RS preferentially aminoacylates an E. coli tyrosyl tRNA (Ec-tRNATry) that recognizes the amber selector codon, and
b. contacting the cell culture medium with one, or more, sulfotyrosine residues under conditions suitable for incorporation of the one, or more, sulfotyrosine residues into the protein or peptide at the sites of the selector codon(s),
thereby producing the protein or peptide of interest with one, or more site-specifically incorporated sulfotyrosine residues.

13. The method of claim 12, wherein the variant E. coli tyrosyl-tRNA synthetase (EcTyr-RS) preferentially aminoacylates an E. coli tyrosyl-tRNA (EctRNAtyr) with a tyrosine analog over the naturally-occurring tyrosine amino acid, wherein the variant EcTyr-RS comprises the amino acid sequence of SEQ ID NO: 1 or an amino acid sequence with at least 90% sequence identity with the full-length SEQ ID NO: 1, wherein the EcTyr-RS variant is mutated, relative to SEQ ID NO: 1, such that the leucine (L) at position 71 is replaced with valine (V), the aspartic acid (D) at position 182 is replaced with G, the phenylalanine at position 183 is either conserved or mutated to Y, and the L at position 186 is either conserved, or is replaced with M, I, or V.

14. The method of claim 12, wherein the Ec-tRNAtyr polynucleotide sequence comprises SEQ ID NO: 10, or a homologous bacteria-derived tRNA comprising at least about 80% sequence identity with SEQ ID NO: 10, wherein the tRNA has an anticodon loop comprising a sequence that specifically binds to a selector sequence of an mRNA, wherein the selector sequence is an amber codon.

15. The method of claim 12, wherein the cell is an E. coli cell or a eukaryotic cell.

16. The method of claim 15, wherein the eukaryotic cell is a mammalian cell.

17. The method of claim 16, wherein the E. coli cell is the ATMY4 strain of E. coli cell.

18. The method of claim 12, wherein the cell further comprises a second tRNA/RS pair that is orthogonal to the cell, wherein the second pair does not cross-react with the EcTyr-RS/tRNA pair and that recognizes an amber selector codon in the protein, wherein the protein or peptide of interest produced contains one, or more sulfotyrosyl residues and one, or more, distinct unnatural amino acid residues other than a sulfotyrosyl residue.

19. A kit for producing a protein or peptide of interest in a cell, wherein the protein or peptide comprises one, or more tyrosyl analogs at specific sites in the protein or peptide, the kit comprising:
a. a container containing a nucleotide sequence encoding an Ec-tRNA^{tyr} that recognizes an amber selector codon in a nucleic acid encoding the protein or peptide of interest, wherein the nucleic acid encoding the protein or peptide incorporates one, or more amber selector codons at specific sites; and
b. a container containing a variant E. coli tyrosyl-tRNA synthetase of claim 1.

20. The kit of claim 19, wherein the variant EcTry-RS comprises an amino acid sequence selected from the group consisting of: SEQ ID NOS: 4-9.

21. The kit of claim 19, wherein the Ec-tRNATry polynucleotide sequence comprises SEQ ID NO: 10, or a homologous bacteria-derived tRNA comprising at least about 80% sequence identity with SEQ ID NO: 10, wherein the tRNA has an anti-codon loop comprising a sequence that specifically binds to a selector sequence of an mRNA, wherein the selector sequence is an amber codon.


SEQUENCE LISTING (34 sequences)

SEQ ID NO: 1 - protein, 424 aa, Escherichia coli
MASSNLIKQLQERGLVAQVTDEEALAERLAQGPIALYCGFDPTADSLHLGHLVPLLCLKRFQQAGHKPVALVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYDFACLNKQYGVVLQIGGSDQWGNITSGIDLTRRLHQNQVFGLTVPLITKADGTKFGKTEGGAVWLDPKKTSPYKFYQFWINTADADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 2 - protein, 12 aa, synthetic construct
  REGION 1..12: note = Description of sequence: artificial peptide
FLIMVSTAYHCG

SEQ ID NO: 3 - protein, 9 aa, synthetic construct
  REGION 1..9: note = Description of sequence: artificial peptide
NSPTACGDH

SEQ ID NO: 4 - protein, 424 aa, synthetic construct
  REGION 1..424: note = Description of sequence: artificial polypeptide
MASSNLIKQLQERGLVAQVTDEEALAERLAQGPIALYCGFDPTADSLHLGHLVPLLCLKRFQQAGHKPVAVVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYGFACLNKQYGVVLQIGGSDQWGNITSGIDLTRRLHQNQVFGLTVPLITKADGTKFGKTEGGAVWLDPKKTSPYKFYQFWINTADADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 5 - protein, 424 aa, synthetic construct
  REGION 1..424: note = Description of sequence: artificial polypeptide
MASSNLIKQLQERGLVAQVTDEEALAERLAQGPIALYCGFDPTADSLHLGHLVPLLCLKRFQQAGHKPVAVVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYGFACMNKQYGVVLQIGGSDQWGNITSGIDLTRRLHQNQVFGLTVPLITKADGTKFGKTEGGAVWLDPKKTSPYKFYQFWINTADADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 6 - protein, 424 aa, synthetic construct
  REGION 1..424: note = Description of sequence: artificial polypeptide
MASSNLIKQLQERGLVAQVTDEEALAERLAQGPIALYCGFDPTADSLHLGHLVPLLCLKRFQQAGHKPVAVVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYGYACINKQYGVVLQIGGSDQWGNITSGIDLTRRLHQNQVFGLTVPLITKADGTKFGKTEGGAVWLDPKKTSPYKFYQFWINTADADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 7 - protein, 424 aa, synthetic construct
  REGION 1..424: note = Description of sequence: artificial polypeptide
MASSNLIKQLQERGLVAQVTDEEALAERLAQGPIALYCGFDPTADSLHLGHLVPLLCLKRFQQAGHKPVAVVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYGYACLNKQYGVVLQIGGSDQWGNITSGIDLTRRLHQNQVFGLTVPLITKADGTKFGKTEGGAVWLDPKKTSPYKFYQFWINTADADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 8 - protein, 424 aa, synthetic construct
  REGION 1..424: note = Description of sequence: artificial polypeptide
MASSNLIKQLQERGLVAQVTDEEALAERLAQGPIALYCGFDPTADSLHLGHLVPLLCLKRFQQAGHKPVAVVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYGYACLNKQYGVVLQIGGSDQWGNITSGIDLTRRLHQNQVFGLTVPLITKADGTKFGKTEGGAVWLDPKKTSPYKFYQFWINTARADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 9 - protein, 424 aa, synthetic construct
  REGION 1..424: note = Description of sequence: artificial polypeptide
MASSNLIKQLQERGLVAQVTDEEALAERLAQGPIALYCGFDPTADSLHLGHLVPLLCLKRFQQAGHKPVAVVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYGFACVNKQYGVVLQIGGSDQWGNITSGIDLTRRLHQNQVFGLTVPLITKADGTKFGKTEGGAVWLDPKKTSPYKFYQFWINTADADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 10 - DNA, 82 bp, Escherichia coli
  source 1..82: mol_type = genomic DNA
ggtggggttcccgagcggccaaagggagcagactctaaatctgccgtcacagacttcgaaggttcgaatccttcccccacca

SEQ ID NO: 11 - protein, 23 aa, synthetic construct
  REGION 1..23: note = Description of sequence: artificial peptide
ENTVTNDWIPEGEEDDDYLDLEK

SEQ ID NO: 12 - skipped

SEQ ID NO: 13 - protein, 11 aa, synthetic construct
  REGION 1..11: note = Description of sequence: artificial peptide
FSEDDDYIDIV

SEQ ID NO: 14 - skipped

SEQ ID NO: 15 - DNA, 12823 bp, synthetic construct
  misc_feature 1..12823: note = Description of sequence: artificial polynucleotide
  source 1..12823: mol_type = other DNA
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacgtacccgtagtggctatggcagggcttgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagttatttccagcaaatcagacagtaattctttttaccgcgacgcagtaaggtaaaacgaccaaacagacgatcttcttctttaaagaagtattcaggatcggactgtttttcaccgttaatggtgatggcattggaggcgatagttttacgtgcctgaccacgggaaggttgcagttcagaatcgaccagtgcctgcatcaggtctgcgcccttttccatctcaaccatcggtacgccgtcctgcgccagctgttcgaagtccgcttcactcagcgcactcaaagaaccgctgaacaggcattcggtaatacgttttgccgcctgtaaaccttcttcaccgtgaaccagacgagtcacctgctccgccagtacatactgggcgcgcggtgctttaccgctgtttttatcttcttcttccagggcgttgatctcttcaatgctcataaaggtgaagaacttcaggaagcggtaaacgtcggcatccgcagtgttgatccagaactggtagaatttgtacgggctggttttcttcggatccaaccagactgcgccgccttcagttttaccaaatttggtgccatctgctttagtgatcagcggaacggtcaggccaaacacctgattctgatgcagacgacgggtcaggtcgataccagaagtgatgttaccccactggtcagaaccaccaatttgcagcaccacaccgtactgtttgttcaaacaggccatactataaccctgcagcaggttgtaggaaaactcagtgaacgaaatcccctgatcttcacggttgagacgctgcttaaccgcttctttgttgatcatctggttaacggagaagtgtttgccaatatcgcgcaggaaggtcagcacattcatattgccgaaccagtcataattattggccgcgatagcagagttttctccacagtcgaaatcgaggaacggggcaacctgcttacggattttgtccacccactcctgaacagtttcttcggtgttcagcttacgctcggcagctttgaagctcgggtcgccaatcagacccgtcgcgccgcctaccagcgcaaccggcttgtggcccgcctgctggaagcgtttcaggcataacaatggaacaagatgccccaaatgcaagctgtcagcggtaggatcgaagccacacacgagtgcgatcgggccttgcgccagtcgctctgctaacgcttcctcgtccgtcacctgggctaccagcccccgctcttgcaattgtttaatcaagttactgcttgccatggtggcgctagccagcttgggtctccctatagtgagtcgtattaatttcgataagccagtaagcagtgggttctctagttagccagagagctctagaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccttcgaacggccactagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagtcaataatcaatgtcaacgcgtatatctggcccgtacatcgcgaagcagcgcaaaacggatcctgcaggtatttgcggccgcggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcgaactcctaaaaaaccgccaccatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtgataaggccaggccggccaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 16 - DNA, 17697 bp, synthetic construct
  misc_feature 1..17697: note = Description of sequence: artificial polynucleotide
  source 1..17697: mol_type = other DNA
cctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggctacgtacccgtagtggctatggcagggcttgcgcttaatgcgccgctacagggcgcgtggggataccccctagagccccagctgaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgagatctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctctagactcgagttaaagtcgacttaacgcgttgaattcttaaacgggccctttccagcaaatcagacagtaattctttttaccgcgacgcagtaaggtaaaacgaccaaacagacgatcttcttctttaaagaagtattcaggatcggactgtttttcaccgttaatggtgatggcattggaggcgatagttttacgtgcctgaccacgggaaggttgcagttcagaatcgaccagtgcctgcatcaggtctgcgcccttttccatctcaaccatcggtacgccgtcctgcgccagctgttcgaagtccgcttcactcagcgcactcaaagaaccgctgaacaggcattcggtaatacgttttgccgcctgtaaaccttcttcaccgtgaaccagacgagtcacctgctccgccagtacatactgggcgcgcggtgctttaccgctgtttttatcttcttcttccagggcgttgatctcttcaatgctcataaaggtgaagaacttcaggaagcggtaaacgtcggcatccgcagtgttgatccagaactggtagaatttgtacgggctggttttcttcgggtccaaccagactgcgccgccttcagttttaccaaatttggtgccatctgctttagtgatcagcggaacggtcaggccaaacacctgattctgatgcagacgacgggtcaggtcgataccagaagtgatgttaccccattggtcagaaccaccaatttgcagcaccacaccgtactgtttgttagcacaggccataccataaccctggagcaggttgtaggaaaactcagtgaacgaaatcccctgaccttcacggttgagacgctgcttaaccgcttctttgttgatcatctggttaacggagaagtgtttgccaatatcgcgcaggaaggtcagcacattcatattgccgaaccagtcataattattggccgcgatagcagagttttctccacagtcgaaatcgaggaacggggcaacctgcttacggattttgtccacccactcctgaacagtttcttcggtgttcagcttacgctcggcagctttgaagctcgggtcgccaatcagacccgtcgcgccgcctaccagcgcaaccggcttgtggcccgcctgctggaagcgtttcaggcataacaatggaacaagatgccccaaatgcaagctgtcagcggtaggatcgaagccacaaatgagtgcgatcgggccttgcgccagtcgctctgctaacgcttcctcgtccgtcacctgggctaccagcccccgctcttgcaattgtttaatcaagttactgcttgccatggtggcgctagccagcttgggtctccctatagtgagtcgtattaatttcgataagccagtaagcagtgggttctctagttagccagagagctctagaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccttcgaacggccactagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagcaaaaaatggagggggacggattcgaaccgccgaacccaaagggagcggatttagagtccgccgcgtttagccacttcgctacccctccggtgtctctatcactgatagggaacttataagtctctatcactgatagggatttcacgtttatggtgatttcccagaacacatagcgacatgcaaatattaaaaaatggtgggggaaggattcgaaccttcgaagtctgtgacggcagatttagagtctgctccctttggccgctcgggaaccccaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgacctagtcaataatcaatgtcaacgcgtatatctggcccgtacatcgcgaagcagcgcaaaacggatcctgcaggtatttgcggccgcggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcgaactcctaaaaaaccgccaccatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtgataagtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggccaaggaggccaccatgaaacactcattaaacgcacttctcattttcctcatcataacatctgcgtggggtgggagcaaaggcccgctggatcagctagagaaaggaggggaaactgctcagtctgcagatccccagtgggagcagttaaataacaaaaacctgagcatgcctcttctccctgccgacttccacaaggaaaacaccgtcaccaacgactggattccagagggggaggaggacgacgactatctggacctggagaagatattcagtgaagacgacgactacatcgacatcgtcgacagtctgtcagtttccccgacagactctgatgtgagtgctgggaacatcctccagctttttcatggcaagagccggatccagcgtcttaacatcctcaacgccaagttcgctttcaacctctaccgagtgctgaaagaccaggtcaacactttcgataacatcttcatagcacccgttggcatttctactgcgatgggtatgatttccttaggtctgaagggagagacccatgaacaagtgcactcgattttgcattttaaagactttgttaatgccagcagcaagtatgaaatcacgaccattcataatctcttccgtaagctgactcatcgcctcttcaggaggaattttgggtacacactgcggtcagtcaatgacctttatatccagaagcagtttccaatcctgcttgacttcaaaactaaagtaagagagtattactttgctgaggcccagatagctgacttctcagaccctgccttcatatcaaaaaccaacaaccacatcatgaagctcaccaagggcctcataaaagatgctctggagaatatagaccctgctacccagatgatgattctcaactgcatctacttcaaaggatcctgggtgaataaattcccagtggaaatgacacacaaccacaacttccggctgaatgagagagaggtagttaaggtttccatgatgcagaccaaggggaacttcctcgcagcaaatgaccaggagctggactgcgacatcctccagctggaatacgtggggggcatcagcatgctaattgtggtcccacacaagatgtctgggatgaagaccctcgaagcgcaactgacaccccgggtggtggagagatggcaaaaaagcatgacaaacagaactcgagaagtgcttctgccgaaattcaagctggagaagaactacaatctagtggagtccctgaagttgatggggatcaggatgctgtttgacaaaaatggcaacatggcaggcatctcagaccaaaggatcgccatcgacctgttcaagcaccaaggcacgatcacagtgaacgaggaaggcacccaagccaccactgtgaccacggtggggttcatgccgctgtccacccaagtccgcttcactgtcgaccgcccctttcttttcctcatctacgagcaccgcaccagctgcctgctcttcatgggaagagtggccaaccccagcaggtcccatcatcaccatcatcatcaccaccatcattgaggcctggccggccgccagcacagtggtcgatcgaccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgccctaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggggttcccgagcggccaaagggagcagactctaaatctgccgtcacagacttcgaaggttcgaatccttcccccaccattttttaatatttgcatgtcgctatgtgttctgggaaatcaccataaacgtgaaatccctatcagtgatagagacttataagttccctatcagtgatagagacaccggaggggtagcgaagtggctaaacgcggcggactctaaatccgctccctttgggttcggcggttcgaatccgtccccctccattttttgctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttct

SEQ ID NO: 17 - DNA, 23 bp, synthetic construct
  misc_feature 1..23: note = Description of sequence: artificial oligonucleotide
  source 1..23: mol_type = other DNA
attacgctgacttgacgggacgg

SEQ ID NO: 18 - DNA, 25 bp, synthetic construct
  misc_feature 1..25: note = Description of sequence: artificial oligonucleotide
  source 1..25: mol_type = other DNA
cgcaaccggcttgtggcccgcctgc

SEQ ID NO: 19 - DNA, 53 bp, synthetic construct
  misc_feature 1..53: note = Description of sequence: artificial oligonucleotide
  variation 26: note = n is a, c, t, g, unknown or other
  source 1..53: mol_type = other DNA
gcaggcgggccacaagccggttgcgnbtgtaggcggcgcgacgggtctgattg

SEQ ID NO: 20 - DNA, 24 bp, synthetic construct
  misc_feature 1..24: note = Description of sequence: artificial oligonucleotide
  source 1..24: mol_type = other DNA
gttcgccgcgatagcagagttttc

SEQ ID NO: 21 - DNA, 59 bp, synthetic construct
  misc_feature 1..59: note = Description of sequence: artificial oligonucleotide
  variation 25..27: note = n is a, c, t, g, unknown or other
  source 1..59: mol_type = other DNA
gaaaactctgctatcgcggcgaacnnntatgactggttcggcaatatgaatgtgctgac

SEQ ID NO: 22 - DNA, 28 bp, synthetic construct
  misc_feature 1..28: note = Description of sequence: artificial oligonucleotide
  source 1..28: mol_type = other DNA
gagatcatgtaggcctgataagcgtagc

SEQ ID NO: 23 - DNA, 22 bp, synthetic construct
  misc_feature 1..22: note = Description of sequence: artificial oligonucleotide
  source 1..22: mol_type = other DNA
gctagcgccaccatggcaagca

SEQ ID NO: 24 - DNA, 48 bp, synthetic construct
  misc_feature 1..48: note = Description of sequence: artificial oligonucleotide
  source 1..48: mol_type = other DNA
aataatctcgagttatttccagcaaatcagacagtaattctttttacc

SEQ ID NO: 25 - DNA, 53 bp, synthetic construct
  misc_feature 1..53: note = Description of sequence: artificial oligonucleotide
  source 1..53: mol_type = other DNA
tggcaaagaattggccaaggaggccaccatgaaacactcattaaacgcacttc

SEQ ID NO: 26 - DNA, 50 bp, synthetic construct
  misc_feature 1..50: note = Description of sequence: artificial oligonucleotide
  source 1..50: mol_type = other DNA
tggcggccggccaggcctcaatgatggtggtgatgatgatggtgatgatg

SEQ ID NO: 27 - DNA, 58 bp, synthetic construct
  misc_feature 1..58: note = Description of sequence: artificial oligonucleotide
  source 1..58: mol_type = other DNA
gtcgtcgtcttcactgaatatcttctccaggtccagaaagtcgtcgtcctcctccccc

SEQ ID NO: 28 - DNA, 58 bp, synthetic construct
  misc_feature 1..58: note = Description of sequence: artificial oligonucleotide
  source 1..58: mol_type = other DNA
gtcgtcgtcttcactgaatatcttctccaggtccagctagtcgtcgtcctcctccccc

SEQ ID NO: 29 - DNA, 60 bp, synthetic construct
  misc_feature 1..60: note = Description of sequence: artificial oligonucleotide
  source 1..60: mol_type = other DNA
ctggacctggagaagatattcagtgaagacgacgactttatcgacatcgtcgacagtctg

SEQ ID NO: 30 - DNA, 60 bp, synthetic construct
  misc_feature 1..60: note = Description of sequence: artificial oligonucleotide
  source 1..60: mol_type = other DNA
ctggacctggagaagatattcagtgaagacgacgactagatcgacatcgtcgacagtctg

SEQ ID NO: 31 - DNA, 36 bp, synthetic construct
  misc_feature 1..36: note = Description of sequence: artificial oligonucleotide
  source 1..36: mol_type = other DNA
ctggacctggagaagatattcagtgaagacgacgac

SEQ ID NO: 32 - DNA, 36 bp, synthetic construct
  misc_feature 1..36: note = Description of sequence: artificial oligonucleotide
  source 1..36: mol_type = other DNA
gtcgtcgtcttcactgaatatcttctccaggtccag

SEQ ID NO: 33 - protein, 6 aa, synthetic construct
  REGION 1..6: note = Description of sequence: synthetic 6xHis tag
HHHHHH

SEQ ID NO: 34 - protein, 10 aa, synthetic construct
  REGION 1..10: note = Description of sequence: synthetic 10xHis tag
HHHHHHHHHH